SEQUENCE LISTING (2 sequences)

SEQ ID NO: 1 - DNA, 28 bp, Artificial Sequence
  Single strand DNA oligonucleotide
  misc_feature (1)..(8): barcode
  misc_feature (1)..(8): N is A, G, C or T
nnnnnnnnagagtttgatcctggctcag

SEQ ID NO: 2 - DNA, 19 bp, Artificial Sequence
  Single strand DNA oligonucleotide
tgctgcctcccgtaggagt

(19) United States
(12) Patent Application Publication
(10) Pub. No.: US 2018/0148770 A1
(43) Pub. Date: May 31, 2018

(54) USE OF MICROBIAL METABOLITES FOR TREATING DISEASES

(71) Applicant: Yeda Research and Development Co. Ltd., Rehovot (IL)

(72) Inventors: Eran ELINAV, Mazkeret Batia (IL); Eran SEGAL, Ramat-HaSharon (IL)

(21) Appl. No.: 15/568,818

(22) PCT Filed: Apr. 27, 2016
(86) PCT No.: PCT/IL2016/050442

§ 371 Date: Oct. 24, 2017

Related U.S. Application Data
(60) Provisional application No. 62/260,561, filed on Nov. 29, 2015.
(60) Provisional application No. 62/153,626, filed on Apr. 28, 2015.

(51) Int. Cl. C12Q 1/689; A61P 1/00; A61K 31/417; A61K 31/132

(52) U.S. Cl. CPC C12Q 1/689; A61K 31/132; A61K 31/417; A61P 1/00

(57) ABSTRACT

A method of determining the therapeutic effect of an agent, such as a metabolite, comprising:
(a) exposing a pathological microbiome to the agent; and
(b) comparing the signature of the pathological microbiome following the exposing with a reference signature of a healthy microbiome, wherein when the signature of the microbiome is statistically significantly similar to the healthy microbiome reference signature, it is indicative that the agent has a therapeutic effect on the microbiome. The agent may be provided to a subject having a pathological microbiome once it has been classified as therapeutic.

### FIELD AND BACKGROUND OF THE INVENTION

The present invention, in some embodiments thereof, relates to the use of microbial metabolites for treating disease and detection thereof for diagnosing diseases.

The human intestine carries a vast and diverse microbial ecosystem that has co-evolved with our species and is essential for human health. Mammals possess an ‘extended genome’ of millions of microbial genes located in the intestine: the microbiome. This multigenomic symbiosis is expressed at the proteomic and metabolic levels in the host and it has therefore been proposed that humans represent a vastly complex biological ‘superorganism’ in which part of the responsibility for host metabolic regulation is devolved to the microbial symbionts. Modern interpretation of the gut microbiome is based on a culture-independent, molecular view of the intestine provided by high-throughput genomic screening technologies. Also, the gut microbiome has been directly implicated in the etiopathogenesis of a number of pathological states as diverse as obesity, circulatory disease, inflammatory bowel diseases (IBDs) and autism. The gut microbiota also influences drug metabolism and toxicity, dietary calorific bioavailability, immune system conditioning and response, and post-surgical recovery. The implication is that quantitative analysis of the gut microbiome and its activities are essential for the generation of future personalized healthcare strategies and that the gut microbiome represents a fertile ground for the development of the next generation of therapeutic drug targets. It also implies that the gut microbiome may be directly modulated for the benefit of the host organism.

The gut microbiota therefore perform a large number of important roles that define the physiology of the host, such as immune system maturation, the intestinal response to epithelial cell injury, and xenobiotic and energy metabolism. In most mammals, the gut microbiome is dominated by four bacterial phyla that perform these tasks: Firmicutes, Bacteroidetes, Actinobacteria and Proteobacteria. The phylotype composition can be specific and stable in an individual, and in a 2-year interval an individual conserves over 60% of phylotypes of the gut microbiome. This implies that each host has a unique biological relationship with its gut microbiota, and by definition that this influences an individual's risk of disease.

Background art includes: www(dot)colitisig(dot)com/treatment-for-colitis-antihistamines, Shimizu et al., Adv Exp Med Biol. 2009; 643:265-71. doi: 10.1007/978-0-387-75681-3_27, US Patent Application No. 20140024132 and US Patent Application No. 20090136454.

### SUMMARY OF THE INVENTION

According to an aspect of some embodiments of the present invention there is provided a method of treating a disease in a subject in need thereof comprising:

(a) analyzing metabolites of the microbiome of the subject;

(b) identifying the metabolites that are differentially produced in the microbiome of the subject as compared with the metabolites produced in a microbiome of a healthy subject; and

(c) administering to the subject a therapeutically effective amount of at least one metabolite which is down-regulated in the microbiome of the subject as compared with a microbiome of a healthy subject or administering to the subject a therapeutically effective amount of an agent which down-regulates a metabolite which is up-regulated in the microbiome of the subject as compared with a healthy subject, thereby treating the disease.

According to an aspect of some embodiments of the present invention there is provided a method of monitoring a treatment of a disease in a test subject:

(a) treating the test subject with at least one metabolite that is produced in a microbiome of the subject;

(b) analyzing a signature of the microbiome of the test subject; and subsequently; and

(c) comparing the microbiome signature of the test subject with the microbiome signature of a healthy subject, wherein an increase in the similarity of the microbiome signature of the test subject with the microbiome signature of the healthy subject following the treating as compared to the similarity of the microbiome signature of the test subject with the microbiome signature of the healthy subject prior to the treating is indicative of an effective treatment.

According to an aspect of some embodiments of the present invention there is provided a method of treating a disease in a subject in need thereof comprising administering a therapeutically effective amount of at least two metabolites to the subject, wherein the amount of metabolites provided is such that the metabolite signature of the microbiome of the subject is made more similar to the metabolite signature of the microbiome of a healthy subject, thereby treating the disease.

According to an aspect of some embodiments of the present invention there is provided a method of treating an inflammatory bowel disease in a subject in need thereof comprising administering to the subject a therapeutically effective amount of an agent which down-regulates an amount and/or activity of a metabolite selected from the group consisting of spermine and putrescine thereby treating the inflammatory bowel disease in the subject.

According to an aspect of some embodiments of the present invention there is provided a method of treating an inflammatory bowel disease in a subject in need thereof comprising co-administering to the subject a therapeutically effective amount of taurine and at least one agent which down-regulates an amount and/or activity of a metabolite selected from the group consisting of histamine, spermine and putrescine thereby treating the an inflammatory bowel disease in the subject.

According to an aspect of some embodiments of the present invention there is provided a method of diagnosing an inflammatory bowel disease in a subject comprising analyzing the amount of a metabolite selected from the group consisting of taurine, histamine, putrescine and spermine produced in the microbiome of the subject, when a decrease in taurine below a predetermined level and/or an increase in histamine, putrescine or spermine above a predetermined level is indicative of the inflammatory bowel disease.

According to an aspect of some embodiments of the present invention there is provided an article of manufacture comprising taurine and an agent that down-regulates an amount and/or activity of a metabolite selected from the group consisting of histamine, spermine and putrescine.

According to an aspect of some embodiments of the present invention there is provided a method of determining the therapeutic effect of an agent:

(a) exposing a pathological microbiome to the agent; and

(b) comparing the signature of the pathological microbiome following the exposing with a reference signature of a healthy microbiome, wherein when the signature of the microbiome is statistically significantly similar to the healthy microbiome reference signature, it is indicative that the agent has a therapeutic effect on the microbiome.

According to some embodiments of the invention, the signature of the microbiome comprises a metabolite signature of the microbiome.

According to some embodiments of the invention, the method further comprises comparing the metabolites that are produced in the microbiome of the subject with the metabolites that are produced in the microbiome of a diseased subject.

According to some embodiments of the invention, the microbiome is selected from the group consisting of a gut microbiome, an oral microbiome, a bronchial microbiome, a skin microbiome and a vaginal microbiome.

According to some embodiments of the invention, the microbiome is a gut microbiome.

According to some embodiments of the invention, the analyzing is effected in a fecal sample of the subject.

According to some embodiments of the invention, the analyzing is effected in a blood sample of the subject.

According to some embodiments of the invention, the metabolite signature comprises a dynamic metabolite signature.

According to some embodiments of the invention, the disease is an inflammatory disease.

According to some embodiments of the invention, the disease is a metabolic disease.

According to some embodiments of the invention, the inflammatory disease is inflammatory bowel disease.

According to some embodiments of the invention, the inflammatory bowel disease is colitis.

According to some embodiments of the invention, the inflammatory bowel disease is Crohn's disease.

According to some embodiments of the invention, the metabolic disease is Diabetes or pre-Diabetes.

According to some embodiments of the invention, the taurine and the agent are coformulated in a single composition.

According to some embodiments of the invention, the taurine and the agent are formulated in individual compositions.

According to some embodiments of the invention, the agent is a metabolite.

According to some embodiments of the invention, the signature is a metabolic signature.

According to some embodiments of the invention, the method further comprises analyzing the microbiome signature of the subject prior to step (a).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-N. Microbiota activation of inflammasome signaling results in downstream induction of antimicrobial peptides.

(A, B) Immunoblot analysis (A) and quantification (B) of pro-caspase-1 (p45) and cleaved caspase-1 (p20) in colon tissue from germ-free and conventionalized (SPF) mice.

(C-E) IL-18 production by colon explants from germ-free (GF) mice (C), antibiotics-treated mice (D), and during early stages of post-natal colonization (E).

(F) Differential expression between wild-type (WT) and Il18^{-/-} mice of antimicrobial peptides (AMPs) versus all other genes. Box=interquartile range (IQR)=25th to 75th percentile, line—median, star—mean, whiskers—IQR*1.5. Mann-Whitney U-test p<0.0001.

(G) Heatmap representation of relative expression of AMPs from transcriptome analysis of colon tissue between WT and Il18^{-/-} mice.

(H-K) Expression levels of the indicated AMPs in WT and Il18^{-/-} colonic tissue.

(L-M) Expression levels of the indicated AMPs in IL-18 injected germ-free mice.

(N) Expression levels of Ang4 in colonic explants cultured with or without the NF-κB inhibitor Bay 11-7085.

Data are expressed as mean±SEM. * p<0.05; ** p<0.01; *** p<0.001.

Pairwise comparison was performed using Student's t test, unless stated otherwise.

Results shown are representative of two independent repeats (n=3-13 per group).

FIGS. 2A-O. NLRP6 inflammasome signaling is required for IL-18 production upstream of the induction of AMPs.

(A, C) Differential expression between WT and Asc^{-/-} (A) or Nlrp6^{-/-} (C) mice of AMPs versus all other genes. Box=interquartile range (IQR)=25th to 75th percentile, line—median, star—mean, whiskers—IQR*1.5. Mann-Whitney U-test p<0.05 (A), p<0.005 (C).

(B, D) Heatmap representation of AMPs from transcriptome analysis of colon tissue between WT and Asc^{-/-} (B) or Nlrp6^{-/-} (D) mice.

(E) IL-18 production by colon explants from WT, Asc^{-/-}, IL18^{-/-}, Casp1/11^{-/-}, Nlrp6^{-/-} and Nlrp3^{-/-} mice.

(F) Colonic Ang4 production in WT, Asc^{-/-}, IL18^{-/-}, Casp1/11^{-/-}, Nlrp6^{-/-} and Nlrp3^{-/-} mice.

(G, H) Targeted mass spectrometry of Ang4 peptides identified in fecal samples obtained from WT or Asc^{-/-} mice.

(I-M) Colonic expression of the indicated AMPs in Nlrp6^{-/-}, Asc^{-/-}, and Casp1/11^{-/-} mice following injection of IL-18.

(N) IL-18 production by colon explants from WT and Nlrp6^{-/-} mice (GF and colonized).

(O) Colonic Ang4 expression levels in germ-free Nlrp6^{-/-} mice, germ-free Nlrp6^{-/-} mice injected with PBS, and germ-free Nlrp6^{-/-} mice injected with IL-18.

Data are expressed as mean±SEM. * p<0.05; ** p<0.01; *** p<0.001.

Pairwise comparison was performed using Student's t test, unless stated otherwise.

Results shown are representative of two independent repeats (n=3-14 per group).

FIGS. 3A-L. The inflammasome-antimicrobial peptide axis regulates intestinal microbial community composition.

(A) Principal coordinate analysis (PCoA) of UniFrac distances based on fecal 16S rRNA analysis from ex-germ free Nlrp6^{-/-} mice at different time points following colonization, and Nlrp6^{-/-} mice born and maintained in an SPF vivarium. Trajectory of the colonization time-course is indicated by an arrow.

(B) UniFrac distances between different stages of ex-germ free Nlrp6^{-/-} mouse colonization and SPF Nlrp6^{-/-} mice.

(C) PCoA of UniFrac distances between colonized ex-germ free Nlrp6^{-/-} mice and ex-GF WT mice.

(D-F) PCoA of UniFrac distances based on fecal 16S rRNA analysis from WT, Nlrp6^{-/-}, Asc^{-/-}, and Casp1/11^{-/-} mice injected with IL-18, compared to WT control groups.

(G) Relative UniFrac distance of microbiota between WT and Casp1/11^{-/-} mice with or without injection of IL-18.

(H) Alpha diversity rarefaction of microbiota from WT and Casp1/11^{-/-} mice with or without injection of IL-18.

(I) Heatmap representation of OTU abundance observed in WT and Casp1/11^{-/-} mice with or without injection of IL-18.

(J) Relative UniFrac distance of anaerobically cultured microbiota between WT and Asc^{-/-} mice with or without supplementation of Ang4.

(K) Alpha diversity rarefaction of anaerobically cultured microbiota from WT and Asc^{-/-} mice with or without supplementation of Ang4.

(L) Relative UniFrac distance of anaerobically cultured microbiota with or without supplementation of Ang4.

Data are expressed as mean±SEM. * p<0.05; ** p<0.01; *** p<0.001.

Pairwise comparison was performed using Student's t test, all other comparisons were performed using ANOVA.

Results shown are representative of two independent repeats (n=2-4), panels J-L represent a single repeat (n=4 per group).

FIGS. 4A-L. Dominant takeover of the dysbiotic microbiota upon cohabitation is mediated by suppression of inflammasome activity.

Colonized or germ-free WT mice were cohoused with WT mice or Asc^{-/-} mice for 4 weeks before analysis, designated crWT(WT) and crWT(Asc^{-/-}) when the recipients were colonized, and grWT(WT) and grWT(Asc^{-/-}), when the recipients were germ-free.

(A) Schematic illustration demonstrating cohousing settings. WT or Asc^{-/-} mice served as microbiota donors to either colonized WT recipients or germ-free WT recipients. In this setting, genetically identical mice harbor distinct microbiota configuration.

(B) IL-18 production by colon explants from WT and Asc^{-/-} mice, as well as their respective cohousing partners (crWTs) after culture for 24 hours.

(C) Principal coordinate analysis (PCoA) of UniFrac distances based on fecal 16S rRNA analysis.

(D) Alpha diversity rarefaction of microbiota from WT and Asc^{-/-} mice, as well as their respective cohousing partners (grWTs).

(E) IL-18 production by colon explants from WT and Asc^{-/-} mice, as well as their respective cohousing partners (grWTs) after culture for 24 hours.

(F, G) Immunoblot analysis (F) and quantification (G) of pro-caspase-1 (p45) and cleaved caspase-1 (p20) in colon tissue.

(H) Differential expression between grWT(WT) and grWT(Asc^{-/-}) mice of AMPs versus all other genes. Box=interquartile range (IQR)=25th to 75th percentile, line—median, star—mean, whiskers—IQR*1.5. Mann-Whitney U-test p<0.0001.

(I) Colonic transcript levels of Ang4 in WT and Asc^{-/-} mice, as well as their respective cohousing partners (grWTs).

(J) PCoA of fecal microbiota from grWT(Asc^{-/-}) mice that were injected with either PBS or IL-18, followed by a fecal microbiota analysis.

(K) Heatmap representation of OTU abundance observed in microbiota from grWT(Asc^{-/-}) mice that were injected with either PBS or IL-18.

(L) UniFrac distance observed in microbiota from grWT(Asc^{-/-}) mice that were injected with either PBS or IL-18.

Data are expressed as mean±SEM. * p<0.05; ** p<0.01; *** p<0.001.

Pairwise comparison was performed using Student's t test, unless stated otherwise.

Results shown in panels C-I are representative of 4 independent repeats (n=3-6 per group). Results shown in panel E are representative of two independent repeats (n=4-6 per group).

FIGS. 5A-P: Microbiota metabolites modulate NLRP6 inflammasome signaling in the healthy and dysbiotic settings.

(A) Heatmap representation of differentially abundant microbial modules between grWT(WT) and grWT(Asc^{-/-}) mice. Displayed are significant FDR-corrected genes, Mann-Whitney U-test p<0.05.

(B) Heatmap representation of metabolite abundance in grWT(WT) and grWT(Asc^{-/-}) mice. Displayed are FDR-corrected metabolites, Mann-Whitney U-test p<0.1.

(C) Metabolite screen for induction of IL-18 production by cultured WT colon explants.

(D) IL-18 production by WT colon explants cultured with increasing doses of taurine.

(E, F) Immunoblot (E) and quantification (F) of pro-caspase-1 (p45) and cleaved caspase-1 (p20) in colon tissue following 12 hours incubation with taurine.

(G) Ang4 expression in WT explants cultured with taurine for 8 hours.

(H) IL-18 production by WT and Nlrp6^{-/-} colon explants cultured with taurine.

(I) Metabolite screen for IL-18 suppression by cultured WT colon explants.

(J, K) IL-18 production by colon explants cultured with increasing doses of histamine (J) or spermine (K).

(L-O) Immunoblot analysis (L, N) and quantification (M, O) of pro-caspase-1 (p45) and cleaved caspase-1 (p20) in colon tissue following 12 hours incubation with either histamine (L, M) or spermine (N, O).

(P) IL-18 production by colon explants cultured with histamine and spermine, with or without addition of taurine.

Data are expressed as mean±SEM. * p<0.05; ** p<0.01; *** p<0.001; n.s. not significant.

Pairwise comparison was performed using Student's t test, unless stated otherwise.

Results shown are representative of 2-5 independent repeats (n=4-16 per group).

FIGS. 6A-O. Microbiota metabolites are functionally involved in inflammasome modulation.

(A, B) Immunoblot analysis (A) and quantification (B) of pro-caspase-1 (p45) and cleaved caspase-1 (p20) in colon tissue obtained from WT mice drinking taurine for 14 days.

(C) IL-18 production by colon explants obtained from WT mice drinking taurine for 14 days.

(D) Ang4 expression in sorted epithelial cells (CD45⁻EpCAM⁺) obtained from WT mice drinking taurine for 14 days.

(E-H) WT germ-free mice were drinking taurine and on day 7 administered with LPS. IL-18 expression in the colon (E), immunoblot quantifications of pro-caspase-1 (p45) and cleaved caspase-1 (p20) (F), IL-18 production by colon explants (G), colonic Ang4 expression (H).

(I) IL-18 production by colon explants from WT, Asc^{-/-} or Nlrp6^{-/-} mice drinking taurine for 14 days.

(J, K) Immunoblot analysis (J) and quantification (K) of pro-caspase-1 (p45) and cleaved caspase-1 (p20) in colon tissue obtained from WT mice drinking histamine with or without spermine for 14 days.

(L-O) PCoA (M, N) and relative distance (L, O) of fecal microbiota from WT, Nlrp6^{-/-} and Asc^{-/-} mice administered with taurine.

Data are expressed as mean±SEM. * p<0.05; ** p<0.01; *** p<0.001; n.s. not significant.

Pairwise comparison was performed using Student's t test, unless stated otherwise.

Results shown are representative of 2-5 independent repeats (n=4-16 per group). A-D represent a single experiment.

FIGS. 7A-L. Restoration of the inflammasome-antimicrobial peptide axis ameliorates colitis.

(A) PCoA of colonic mucosal-adherent microbiota from WT mice drinking taurine.

(B) Electron microscopy images of epithelial associated bacteria from WT mice drinking taurine.

(C-F) Acute DSS colitis (1.5% DSS) was induced in antibiotics-treated WT mice with or without administration of 1% taurine in the drinking water. Weight loss (C), colonoscopy severity score on day 7 (D), representative histology images on day 11 (E), pathology scoring on day 11 (F).

(G) Mortality following 2% DSS in WT mice administered with taurine (n=10 mice in each group).

(H, I) Acute DSS colitis (1.5% DSS) was induced in Asc^{-/-} mice with or without administration of 1% taurine in the drinking water. Weight loss (H), colonoscopy severity score on day 7 (I).

(J-L) Acute DSS colitis (1.5% DSS) was induced in Asc^{-/-} mice with or without daily administration of IL-18 for 5 days before induction of colitis. Weight loss (J), representative colonoscopy images (K), colonoscopy severity score on day 7 (L).

Data are expressed as mean±SEM. * p<0.05; ** p<0.01; *** p<0.001; n.s. not significant.

Pairwise comparison was performed using Student's t test, unless stated otherwise.

Results shown in panels A, C-L represent 2-5 independent repeats (n=5-10 per group).

Images shown in panel B are representative of 24 randomly taken electron micrographs.

FIGS. 8A-K. Microbiota induction of inflammasome signaling.

(A-C) IL-18 mRNA levels in germ-free mice (A), antibiotics-treated mice (B), and during early states of post-natal colonization (C).

(D) Gene ontology annotation of genes differentially expressed more than 2-fold between colonic tissue of Il18^{-/-} compared to WT mice. Displayed are FDR-corrected p-values obtained from hypergeometric testing for enrichment.

(E-G) Expression levels of Ang4 (E) Itn11 (F) and Retn1b (G) in germ-free mice compared to SPF controls.

(H) Expression levels of Ang4 during post-natal development.

(I) IL-18 production by colon explants of germ-free mice injected with IL-18.

(J) Expression levels of Ang4 in germ-free colonic explants cultured for 5 hours with recombinant IL-18.

(K) Expression levels of TNFa in colon explants incubated with or without NF-κB inhibitor Bay 11-7085.

Data are expressed as mean±SEM. * p<0.05; ** p<0.01; *** p<0.001.

Pairwise comparison was performed using Student's t test, unless stated otherwise.

Results shown are representative of two independent repeats (n=3-13 per group).

FIGS. 9A-L. Necessity for inflammasome signaling upstream of IL-18-induced AMPs.

(A) Differential expression between WT and Asc^{-/-} mice of AMPs versus all other genes. Box=interquartile range (IQR)=25th to 75th percentile, line—median, star—mean, whiskers—IQR*1.5. Mann-Whitney U-test p<0.0001.

(B) Heatmap relative representation of AMPs from transcriptome analysis of colon tissue between WT and Asc^{-/-} mice.

(C, D) Colonic mRNA levels of Retn1b (C) and Ang1 (D) in WT and Asc^{-/-} mice.

(E) Targeted mass spectrometry histograms of the indicated Ang4 peptide on feces from WT and Asc^{-/-} mice.

(F-H) Colonic IL-18 mRNA (F) and protein (G) and Ang4 mRNA (H) in bone marrow chimeras generated from WT and Il18^{-/-} mice.

(I) IL-18 levels in the serum from mice injected with either PBS or IL-18.

(J, K) Colonic IL-18 expression (J) and production (K) in WT and Nlrp6^{-/-} mice (germ-free compared to SPF).

(L) IL-18 secretion by colonic explants from mice injected with either PBS or IL-18.

Data are expressed as mean±SEM. * p<0.05; ** p<0.01; *** p<0.001.

Pairwise comparison was performed using Student's t test, unless stated otherwise.

Results shown are representative of two independent repeats (n=3-14 per group).

FIGS. 10A-O. Cohousing of germ-free mice with WT or Asc^{-/-} mice.

(A) Alpha diversity rarefaction of microbiota from ex-germ free Nlrp6^{-/-} mice at different time points following colonization, compared to Nlrp6^{-/-} mice born and maintained in an SPF vivarium.

(B) UniFrac distance between fecal microbiota from colonized ex-germ free Nlrp6^{-/-} mice and ex-GF WT mice.

(C) Relative abundance of Bacteroidaceae in fecal samples from ex-germ free WT and Nlrp6^{-/-} mice at different time points following colonization.

(D, F) PCoA of fecal microbiota from WT, Asc^{-/-} and Nlrp6^{-/-} mice housed in two different facilities, Weizmann Institute of Science (D), University of Massachusetts (F).

(E, G) weighted UniFrac distance of fecal microbiota from WT, Asc^{-/-} and Nlrp6^{-/-} mice housed in two different facilities, Weizmann Institute of Science (E), University of Massachusetts (G).

(H) PCoA of fecal microbiota from colonized WT mice which were cohoused with colonized WT mice or Asc^{-/-} mice for 4 weeks before analysis, designated crWT(WT) and crWT(Asc^{-/-}), respectively.

(I) Taxonomic analysis of fecal microbiota obtained from WT and Asc^{-/-} mice, as well as their respective cohousing partners (grWTs).

(J-N) Germ-free mice were cohoused with WT mice or Asc^{-/-} mice for 4 weeks before analysis, designated grWT(WT) and grWT(Asc^{-/-}), respectively. Presented are colonic mRNA levels of the indicated genes.

(O) Ang4 expression in colonized WT mice cohoused with either WT or Asc^{-/-} mice for 4 weeks before analysis, designated crWT(WT) and crWT(Asc^{-/-}), respectively.

* p<0.05; ** p<0.01; *** p<0.001; **** p<0.0001.

Results shown are representative of a two independent repeats (n=2-4 per group), panels J-L represent a single repeat (n=4 per group).

FIGS. 11A-K. Metabolite modulators of the NLRP6 inflammasome

(A, B) PCA analysis of KEGG pathways (A) and heatmap of modules (B) from shotgun metagenomics of fecal microbiota from WT, Asc^{-/-} and Nlrp6^{-/-} mice housed in two different facilities. Heatmap elements were chosen to explain PC3.

(C) Candidate metabolites showing elevated abundance in grWT(WT) compared to grWT(Asc^{-/-}) mice.

(D) Differential abundance of tauC by shotgun metagenomics of fecal microbiota from grWT(WT) compared to grWT(Asc^{-/-}) mice.

(E) IL-18 mRNA levels in colonic explants following incubation with either PBS or taurine.

(F) FACS quantification of propidium iodide positive cells obtained from colon explants incubated for 10 hrs with PBS or taurine.

(G) IL-18 produced by colonic explants from Nlrp3^{-/-} mice incubated with taurine for 24 hours.

(H) Candidate metabolites showing diminished abundance in grWT(WT) compared to grWT(Asc^{-/-}) mice.

(I) Differential abundance of enzymes participating in the spermine metabolism pathway in fecal microbiota from grWT(WT) and grWT(Asc^{-/-}) mice.

(J, K) Bacterial contribution to genes mapped to the polyamine biosynthesis pathway (J) and spermidine transport system (K) analyzed by re-mapping metagenomic reads to a bacterial genome database.

Pairwise comparison was performed using Student's t test.

Results shown were performed with an n=4-6 mice per group.

FIGS. 12A-K Analysis of NLRP6 activators and inhibitors

(A) Differential abundance of enzymes participating in the histidine metabolism pathway in fecal microbiota from grWT(WT) and grWT(Asc^{-/-}) mice.

(B-D) Bacterial contribution to genes mapped to the histidine biosynthesis pathway (B), histidine transport system (C), and histidine degradation (D) analyzed by re-mapping of metagenomic reads to a bacterial genome database.

(E) Il18 mRNA levels in colonic explants following incubation with either PBS, spermine or histamine.

(F) FACS quantification of propidium iodide positive cells obtained from colon explants incubated for 10 hrs with either PBS, spermine or histamine.

(G) IL-18 production by colon explants incubated with putrescine or spermine for 24 hrs.

(H) IL-18 production by colon WT, Nlrp3^{-/-}, Nlrp6^{-/-} or Nlrp3/6^{-/-} explants incubated with either PBS, histamine or spermine.

(I, J) IL-18 production by WT (I) and Nlrp6^{-/-} (J) colonic organoids incubated with either taurine or taurine+histamine+spermine.

(K) Representative organoid images from experiments shown in (I) and (J).

Pairwise comparison was performed using Student's t test.

Data are expressed as mean±SEM. * p<0.05; ** p<0.01; *** p<0.001.

Results shown in panels E-K are representative of 2 independent experiments (n=4-6).

FIGS. 13A-K—Niche analysis following metabolite administration.

(A) Il18 expression in the colon of WT mice drinking taurine for 14 days.

(B) Lamina propria FACS analysis of colon tissue from WT mice drinking taurine for 14 days.

(C) Immunoblot analysis of pro-caspase-1 (p45) and cleaved caspase-1 (p20) in colon tissue from germ-free WT mice drinking taurine and administered with LPS compared to SPF mice.

(D) Lamina propria FACS analysis of colon tissue from WT mice drinking either histamine or histamine and spermine for 14 days.

(E, F) PCoA of fecal microbiota (E) and relative distance (F) from WT mice drinking histamine or spermine for 14 days compared to controls.

(G) Heatmap representation of OTU abundance observed in WT, Asc^{-/-} and Nlrp6^{-/-} drinking taurine compared to controls.

(H) PCoA of fecal microbiota incubated with the indicated metabolites under anaerobic culturing.

(I) Heatmap representation of differentially abundant OTUs of mucosal-adherent bacteria from WT mice drinking taurine compared to controls.

(J) Quantification of electron microscopy images of colon tissue from WT mice drinking taurine compared to controls.

(K) PCoA of mucosal adherent bacteria from WT mice drinking either histamine or spermine compared to controls.

Data are expressed as mean±SEM. Pairwise comparison was performed using Student's t test.

* p<0.05; ** p<0.01; *** p<0.001.

Results shown in panels E-I are representative of 2 independent experiments (n=4-6).

FIGS. 14A-O The metabolite-inflammasome-AMP axis regulates bacterial composition and susceptibility to colitis.

(A-E) Acute DSS colitis (1.5% DSS) was induced in WT mice with or without administration of 1% taurine in the drinking water.

(A) Representative colonoscopy images of day 7 of DSS colitis,

(B) FITC-dextran levels in the serum 3 hours after oral gavage (8 mg/ml FITC-dextran) on day 20 of DSS colitis,

(C) Quantification of bacterial numbers per mg of liver tissue on day 20 of DSS colitis,

(D) ZO-1 staining of colonic tissue on day 20 of DSS colitis,

(E) ZO-1 staining quantification on day 20 of DSS colitis.

(F-I) Acute DSS colitis (1.5% DSS) was induced in WT mice with or without administration of 1% taurine in the drinking water, prior or throughout DSS administration.

(F) Weight loss,

(G) Colonoscopy severity score on day 7,

(H) Representative colonoscopy images,

(I) PCoA of fecal microbiota.

(J, K) Acute DSS colitis (2% DSS) was induced in germ-free WT mice with or without administration of 1% taurine in the drinking water. Germ-free mice that received neither taurine nor DSS served as controls.

(J) Colonoscopy severity score on day 7,

(K) Representative colonoscopy images.

(L, M) Nlrp6^{-/-} mice were given 1.5% DSS in the drinking water, with or without supplementation of taurine in the drinking water.

(L) Colonoscopy severity score on day 7,

(M) Weight loss.

(N, O) Acute DSS colitis (1.5% DSS) was induced in WT mice with or without administration of histamine and antibiotics in the drinking water,

(N) Colonoscopy severity score on day 7,

(O) Representative colonoscopy images.

Pairwise comparison was performed using Student's t test.

Data are expressed as mean±SEM. * p<0.05; ** p<0.01; *** p<0.001.

Results shown are representative of 2 independent experiments (n=4-8). F-I represent one experiment (n=5).

### DESCRIPTION OF SPECIFIC EMBODIMENTS OF THE INVENTION

The present invention, in some embodiments thereof, relates to the use of microbial metabolites for treating disease and detection thereof for diagnosing diseases in general and inflammatory diseases in particular.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Host-microbiome co-evolution drives homeostasis and disease susceptibility, yet regulatory principles governing intestinal niche formation and stability remain unknown. Inflammasome signaling orchestrates these interactions, but its activators and niche-modulating mechanisms are obscure. The present inventors have identified a set of microbiota metabolites whose integrative activity results in NLRP6 inflammasome signaling during steady-state commensal colonization, leading to downstream epithelial IL-18-induced anti-microbial peptide (AMP) secretion, thereby enabling the microbiota to modulate a host immune pathway, and to induce favorable conditions for its own colonization. The present inventors show that a disruption in inflammasome signaling leads to development of an altered AMP program, leading to development of dysbiosis (FIGS. 2A-D). Furthermore, they demonstrate that the resultant dysbiotic microbiota configuration acquires inflammasome-suppressive capabilities through an altered metabolite secretion. This acquired dominant trait enables stable colonization of the dysbiotic microbiota in a genetically intact host, by hijacking its niche-promoting innate immune signaling. This leads to an alteration of the invaded host's niche towards one that resembles the invading ecosystem's niche of origin, thereby ensuring its persistent colonization and community stability.

Using an integrated metabolomics-metagenomics approach, the present inventors identified the organic acid taurine as a mucosal inflammasome activator (FIGS. 5G and 6A-B), and the metabolites histamine and spermine as inhibitors of inflammasome activation and IL-18 secretion (FIGS. 6C-H). In corroboration with these results, the present inventors identified (using shotgun metagenomic sequencing) microbial enzymes that could potentially be involved in taurine, histamine and spermine biosynthesis in bacteria (namely glutamate decarboxylase (EC 4.1.1.15) for taurine, ornithine decarboxylase for spermine, and enzymes involved in the histamine pathway for histamine—FIGS. 13A-D).

The results of the experiments performed suggest that microbiota composition and function may be therapeutically exploited by methods different from, and complementary to pro-biotic and pre-biotic approaches. As such, rather than attempting to change the microbiota itself, such “post-biotic” treatment employing or manipulating downstream microbiota-produced or -modulated metabolites, allows for the control of the host-microbiota niche. Such intervention would harness the endogenous physiological forces shaping microbiota composition to drive it from disease-prone towards a healthy configuration. Exploiting common downstream metabolic outputs that are reflective of microbiota activity rather than composition, may circumvent the strong inter-individual variability in microbiota composition that severely limits effective pre- and probiotic treatment. Such novel therapeutic approaches, potentially present opportunities for rational design of microbiota-modulating therapeutics in a variety of multi factorial disorders.

As a proof of concept of intervening with the microbiota component of the niche-promoting co-regulatory pathway, taurine (100 mg/ml) was administered in the drinking water to naïve WT mice for two weeks. Following induction of taurine-induced microbiota alterations (FIG. 12H), DSS colitis was induced, resulting in improved weight loss (FIG. 7A) and ameliorated colitis severity (FIGS. 7B-E, in taurine-treated versus control mice. Furthermore, the taurine-administered group exhibited enhanced survival (FIG. 7F), and improved mucosal barrier integrity as indicated by a reduced systemic FITC-dextran influx, decreased hepatic bacterial load, and sustained epithelial tight junctions integrity (FIGS. 7G, 7H, and 14A).

Thus, according to one aspect of the present invention there is provided a method of treating a disease in a subject in need thereof comprising:

(a) analyzing metabolites of the microbiome of the subject;

(b) identifying the metabolites that are differentially produced in the microbiome of the subject as compared with the metabolites produced in a microbiome of a healthy subject; and

(c) administering to the subject a therapeutically effective amount of at least one metabolite which is down-regulated in the microbiome of the subject as compared with a microbiome of a healthy subject or administering to the subject a therapeutically effective amount of an agent which down-regulates a metabolite which is up-regulated in the microbiome of the subject as compared with a healthy subject, thereby treating the disease.

As used herein, a “metabolite” is an intermediate or product of metabolism. The term metabolite is generally restricted to small molecules and does not include polymeric compounds such as DNA or proteins greater than 100 amino acids in length. A metabolite may serve as a substrate for an enzyme of a metabolic pathway, an intermediate of such a pathway or the product obtained by the metabolic pathway.

According to a particular embodiment, the metabolite is one that alters the composition or function of the microbiome.

In preferred embodiments, metabolites include but are not limited to sugars, organic acids, amino acids, fatty acids, hormones, vitamins, as well as ionic fragments thereof. In another embodiment, the metabolite is an oligopeptides (less than about 100 amino acids in length).

In particular, the metabolites are less than about 3000 Daltons in molecular weight, and more particularly from about 50 to about 3000 Daltons.

Preferably, the metabolite is present in the microbes of the microbiome or secreted from the microbes of the microbiome. Cells can also be lysed in order to measure cellular products present within the cell.

The metabolite of this aspect of the present invention may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention are involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

According to a particular embodiment, the metabolite is selected from the group consisting of taurine, pinitol, sebacate, undecanedioate, dodencanedioate, homoserine, taurodeoxycholate, chenodeoxycholate, tryptamine, glutarate, ethylmalonate, histamine, spermine, AMP, GABA, N-acetyltryptophan, pipecolic acid and N-acetylproline.

As used herein, the term “microbiome” refers to the totality of microbes (bacteria, fungae, protists), their genetic elements (genomes) in a defined environment.

The microbiome may be for example a gut microbiome, an oral microbiome, a bronchial microbiome, a skin microbiome or a vaginal microbiome.

According to a particular embodiment, the microbiome is a gut microbiome (i.e. intestinal microbiome).

As used herein, the term “microbiome metabolome” refers to the complete set of small-molecule metabolites (such as metabolic intermediates, hormones and other signaling molecules, and secondary metabolites) found within a microbiome.

The present invention contemplates analyzing at least one metabolite, at least two metabolites, at least three metabolites, four metabolites, five metabolites, 10 metabolites, 20 metabolites, 50 metabolites, 100 metabolites in order to ascertain which metabolite to provide in order to treat a disease of a subject.

According to one embodiment the sample is frozen and/or lyophilized prior to analysis. According to another embodiment, the sample may be subjected to solid phase extraction methods.

In one embodiment, metabolites are identified using a physical separation method.

The term “physical separation method” as used herein refers to any method known to those with skill in the art sufficient to produce a profile of changes and differences in small molecules produced in hSLCs, contacted with a toxic, teratogenic or test chemical compound according to the methods of this invention. In a preferred embodiment, physical separation methods permit detection of cellular metabolites including but not limited to sugars, organic acids, amino acids, fatty acids, hormones, vitamins, and oligopeptides, as well as ionic fragments thereof and low molecular weight compounds (preferably with a molecular weight less than 3000 Daltons, and more particularly between 50 and 3000 Daltons). For example, mass spectrometry can be used. In particular embodiments, this analysis is performed by liquid chromatography/electrospray ionization time of flight mass spectrometry (LC/ESI-TOF-MS), however it will be understood that metabolites as set forth herein can be detected using alternative spectrometry methods or other methods known in the art for analyzing these types of compounds in this size range.

Certain metabolites can be identified by, for example, gene expression analysis, including real-time PCR, RT-PCR, Northern analysis, and in situ hybridization.

In addition, metabolites can be identified using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), or ion mobility spectrometry (e.g. GC-IMS, IMS-MS, LC-IMS, LC-IMS-MS etc.).

Mass spectrometry methods are well known in the art and have been used to quantify and/or identify biomolecules, such as proteins and other cellular metabolites (see, e.g., Li et al., 2000; Rowley et al., 2000; and Kuster and Mann, 1998).

In certain embodiments, a gas phase ion spectrophotometer is used. In other embodiments, laser-desorption/ionization mass spectrometry is used to identify metabolites. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”).

In MALDI, the metabolite is mixed with a solution containing a matrix, and a drop of the liquid is placed on the surface of a substrate. The matrix solution then co-crystallizes with the biomarkers. The substrate is inserted into the mass spectrometer. Laser energy is directed to the substrate surface where it desorbs and ionizes the proteins without significantly fragmenting them. However, MALDI has limitations as an analytical tool. It does not provide means for fractionating the biological fluid, and the matrix material can interfere with detection, especially for low molecular weight analytes.

In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the biomarker of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte (e.g. biomarker) and adding matrix-containing liquid to the captured analyte to provide the energy absorbing material.

For additional information regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

In some embodiments, the data from mass spectrometry is represented as a mass chromatogram. A “mass chromatogram” is a representation of mass spectrometry data as a chromatogram, where the x-axis represents time and the y-axis represents signal intensity. In one aspect the mass chromatogram is a total ion current (TIC) chromatogram. In another aspect, the mass chromatogram is a base peak chromatogram. In other embodiments, the mass chromatogram is a selected ion monitoring (SIM) chromatogram. In yet another embodiment, the mass chromatogram is a selected reaction monitoring (SRM) chromatogram. In one embodiment, the mass chromatogram is an extracted ion chromatogram (EIC).

In an EIC, a single feature is monitored throughout the entire run. The total intensity or base peak intensity within a mass tolerance window around a particular analyte's mass-to-charge ratio is plotted at every point in the analysis. The size of the mass tolerance window typically depends on the mass accuracy and mass resolution of the instrument collecting the data. As used herein, the term “feature” refers to a single small metabolite, or a fragment of a metabolite. In some embodiments, the term feature may also include noise upon further investigation.

Detection of the presence of a metabolite will typically involve detection of signal intensity. This, in turn, can reflect the quantity and character of a biomarker bound to the substrate. For example, in certain embodiments, the signal strength of peak values from spectra of a first sample and a second sample can be compared (e.g., visually, by computer analysis etc.) to determine the relative amounts of particular metabolites. Software programs such as the Biomarker Wizard program (Ciphergen Biosystems, Inc., Fremont, Calif.) can be used to aid in analyzing mass spectra. The mass spectrometers and their techniques are well known.

A person skilled in the art understands that any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run. Good stable isotopic labeling is included.

In one embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In one embodiment of the invention, levels of metabolites are detected by MALDI-TOF mass spectrometry.

Methods of detecting metabolites also include the use of surface plasmon resonance (SPR). The SPR biosensing technology has been combined with MALDI-TOF mass spectrometry for the desorption and identification of metabolites.

Data for statistical analysis can be extracted from chromatograms (spectra of mass signals) using softwares for statistical methods known in the art. “Statistics” is the science of making effective use of numerical data relating to groups of individuals or experiments. Methods for statistical analysis are well-known in the art.

In one embodiment a computer is used for statistical analysis.

In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis.

In different embodiments signals from mass spectrometry can be transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin (Myers, Classical and Modern Regression with Applications, 2nd edition, Duxbury Press, 1990).

Analyzing metabolites produced in the microbiome may be effected by analyzing a microbiome sample of the subject. Thus, for example stool samples may be taken to analyze the gut microbiome, bronchial samples may be taken to analyze the bronchial microbiome etc. According to a particular embodiment, analyzing the metabolites produced in a microbiome of a subject is determined from a stool sample of the subject.

It will be appreciated that the microbiome source depends on the disease which is being treated. Thus, for example if the disease is related to a gut microbiome (e.g. colitis), then a stool sample may be analyzed. If the disease is related to a bronchial microbiome (e.g. asthma), then a bronchial or phlegm sample may be analyzed.

It will be further appreciated that the metabolites produced in a microbiome may be released from the microbiome. Such metabolites may alter the general metabolome of the subject. Therefore, the present invention also contemplates analyzing the metabolites in a blood sample of the diseased subject or a urine sample of the subject.

The analysis of the metabolites may be a qualitative analysis (e.g. all or none) or a qualitative analysis (i.e. analyzing the level of metabolites).

The term “subject” as used herein, refers to mammals (e.g. humans).

Once the metabolites that are produced in the microbiome of the diseased subject are analyzed, the present invention proposes comparing them with metabolites produced in a microbiome of a healthy subject so as to reveal metabolites that are differentially produced from the microbiome of the diseased subject.

The term “healthy subject” refers to a subject that does not have the disease of the diseased subject. Preferably, the healthy subject does not have any metabolic disease, immune disease or cancerous disease (e.g. is not diabetic or prediabetic, does not have Crohn's disease).

It will be appreciated that metabolites produced in microbiomes of the same source are compared (i.e. metabolites of the gut microbiome of a diseased human subject is compared with metabolites of the gut microbiome of a healthy human subject). Preferably, the diseased subject is the same age and sex as the healthy subject. In addition, if a fecal sample of the diseased subject is analyzed, then a fecal sample of the healthy subject is analyzed etc.

The present inventors have shown that changes in eating patterns (e.g. due to circadian misalignment) affect the composition of the microbiome. Therefore, preferably samples are taken from the diseased subject and the healthy subject at the same time in the day.

According to one embodiment, the metabolite profile of the diseased subject under analysis may be included in a subject specific database, and the metabolite profile of the healthy microbiome derived from a healthy subject may be included in a second database. The second database may comprise metabolite profiles of more than one healthy microbiome and may comprise average data from a plurality of healthy microbiomes.

Both the subject-specific database and the second database may be stored in a computer readable format on a computer readable medium, and is optionally and preferably accessed by a data processor, such as a general purpose computer or dedicated circuitry.

The subject-specific database may comprise additional data describing the subject. Representative examples of types of data other than the metabolite profile or signature include without limitation responses to foods, blood chemistry of the subject, partial blood chemistry of the subject, genetic profile of the subject, microbial data associated with the microbiome of the subject, the medical condition of the subject, sleep patterns of the subject, food intake habits of the subject and the like. The subject-specific database may also comprise data pertaining to the disease of the subject. These and other types of data are described in more detail below.

The method may further include comparing the metabolites that are produced in the microbiome of the subject with the metabolites that are produced in the microbiome of a subject having the same disease as the test subject.

Metabolites that may be considered to be differentially produced in the microbiome of the diseased subject as compared to the healthy subject may be upregulated by at least 1.5 fold, 2 fold, 3 fold, 4 fold, 5 fold, 10 fold or greater or downregulated by at least 1.5 fold, 2 fold, 3 fold, 4 fold, 5 fold, 10 fold or greater.

If a metabolite which is downregulated in the diseased subject as compared to the non-diseased subject is identified, the present invention contemplates administering to the subject a therapeutically effective amount of the metabolite. If a metabolite which is upregulated in the diseased subject as compared to the non-diseased subject is identified, the present invention contemplates administering to the subject a therapeutically effective amount of an inhibitor or antagonist of the metabolite.

As used herein, the phrase “metabolite inhibitor” or “metabolite antagonist” refers to an agent which acts directly or indirectly with the metabolite to down-regulate the activity and/or amount of the metabolite.

According to a particular embodiment, the metabolite inhibitor is not an antibiotic.

The present invention contemplates administering at least one metabolite, two metabolites, three metabolites, four metabolites, five metabolites, at least 10 metabolites, at least 20 metabolites, at least 30 metabolites, at least 40 metabolites, at least 50 metabolites.

According to a particular embodiment, the metabolites are provided as isolated metabolites (i.e. not part of microbial compositions).

The present invention contemplates administering at least one metabolite inhibitor, two metabolite inhibitors, three metabolite inhibitors, four metabolite inhibitors, five metabolite inhibitors, at least 10 metabolite inhibitors, at least 20 metabolite inhibitors, at least 30 metabolite inhibitors, at least 40 metabolite inhibitors, at least 50 metabolite inhibitors.

The present invention further contemplates administering a combination of metabolites and metabolite inhibitors which act in concert to control the host-microbiota niche in a particular direction. Thus, for example, the present inventors propose administering taurine as a metabolite together with an inhibitor of at least one of the following metabolites—histamine, spermine or putrescine for the treatment of an inflammatory bowel disease.

In addition to the metabolites and/or metabolite inhibitors, the present invention further contemplates treating the subject with microbial compositions to drive the subject microbiome from disease-prone towards a healthy configuration.

Other diseases which may be treated according to this aspect of the present invention typically involve microbiome dysbiosis associated diseases.

Exemplary microbiome dysbiosis associated diseases include but are not limited to metabolic diseases and inflammatory diseases.

Metabolic disorders include, but are not limited to, hyperglycemia, prediabetes, diabetes (type I and type 2), obesity, insulin resistance, metabolic syndrome and dyslipidemia due to type 2 diabetes.

As used herein the phrase “inflammatory disorder” includes but is not limited to chronic inflammatory diseases and acute inflammatory diseases. According to a particular embodiment, the disease is an inflammatory bowel disease. Other examples of inflammatory diseases and conditions are summarized infra.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphocytic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

As mentioned, the present inventors propose providing a subject with more than one metabolite or metabolite inhibitor. Thus, according to another aspect of the present invention there is provided a method of treating a disease in a subject in need thereof comprising administering a therapeutically effective amount of at least two metabolites to the subject, wherein the amount of metabolites provided is such that the metabolite signature of the microbiome of the subject is made more similar to the metabolite signature of the microbiome of a healthy subject, thereby treating the disease.

According to still another aspect of the present invention there is provided a method of treating a disease in a subject in need thereof comprising administering a therapeutically effective amount of at least two metabolite inhibitors to the subject, wherein the amount of metabolite inhibitors provided is such that the metabolite signature of the microbiome of the subject is made more similar to the metabolite signature of the microbiome of a healthy subject, thereby treating the disease.

The metabolite signature according to this aspect of the present invention refers to the presence and/or amount of at least two, five, 10, 20, 50, 100, 200 or all the metabolites of the microbiome.

In some embodiments, a metabolite signature includes information relating to presence, level, and/or activity of at least 10% of the metabolites of the microbes of the microbiome. In some embodiments, a metabolite signature includes information relating to presence, level, and/or activity of at least 20% of the metabolites of the microbes of the microbiome. In some embodiments, a metabolite signature includes information relating to presence, level, and/or activity of at least 30% of the metabolites of the microbes of the microbiome. In some embodiments, a metabolite signature includes information relating to presence, level, and/or activity of at least 40% of the metabolites of the microbes of the microbiome. In some embodiments, a metabolite signature includes information relating to presence, level, and/or activity of at least 50% of the metabolites of the microbes of the microbiome. In some embodiments, a metabolite signature includes information relating to presence, level, and/or activity of at least 60% of the metabolites of the microbes of the microbiome. In some embodiments, a metabolite signature includes information relating to presence, level, and/or activity of at least 70% of the metabolites of the microbes of the microbiome. In some embodiments, a metabolite signature includes information relating to presence, level, and/or activity of at least 80% of the metabolites of the microbes of the microbiome. In some embodiments, a metabolite signature includes information relating to presence, level, and/or activity of at least 90% of the metabolites of the microbes of the microbiome. In some embodiments, a metabolite signature includes information relating to presence, level, and/or activity of 100% of the metabolites of the microbes of the microbiome.

The metabolite signature may refer to a metabolite signature at a particular time of day (i.e. the dynamic metabolic signature).

Methods of determining whether two metabolite signatures can be classified as being similar are described herein below.

According to this aspect of the present invention at least 2, at least 5, at least 10, at least 20, at least 50 metabolites are provided to the subject. According to a particular embodiment, the entire metabolome of a healthy microbiome is provided to the subject.

According to this aspect of the present invention at least 2, at least 5, at least 10, at least 20, at least 50 metabolite inhibitors are provided. The inhibitors may be provided alone or in combination with the metabolites.

As mentioned herein above, the present inventors have shown that metabolite taurine is downregulated in the microbiome of a colitis patient, whereas the metabolites histamine, spermine and putrescine are upregulated in the microbiome of a colitis patient.

Thus, according to yet another aspect of the present invention there is provided a method of treating an inflammatory bowel disease in a subject in need thereof comprising administering to the subject a therapeutically effective amount of an agent which down-regulates an amount and/or activity of a metabolite selected from the group consisting of spermine and putrescine thereby treating the inflammatory bowel disease in the subject.

According to still another aspect of the present invention there is provided a method of treating an inflammatory bowel disease in a subject in need thereof comprising co-administering to the subject a therapeutically effective amount of taurine and at least one agent which down-regulates an amount and/or activity of a metabolite selected from the group consisting of histamine, spermine and putrescine thereby treating the an inflammatory bowel disease in the subject.

As used herein, the phrase “inflammatory bowel disease” refers to a group of inflammatory conditions of the colon and small intestine.

The term “colitis” as used herein refers to an acute or chronic inflammation of the colon, in specific embodiments the membrane lining the large bowel. Symptoms of colitis may include abdominal pain, diarrhea, rectal bleeding, painful spasms (tenesmus), lack of appetite, colonic ulcers, fever, and/or fatigue.

According to a particular embodiment, the colitis is not one which is caused by a food allergy.

Exemplary agents that down-regulate histamine include antihistamines.

Non-limiting examples of antihistamine agents suitable for the present invention include chloropheniramine, brompheniramine, dexchloropeniramine, tripolidine, clemastine, diphenhydramine, promethazine, piperazines, piperidines, astemizole, loratadine, levocetirizine dihydrochloride, 4-(4-(bis(4-fluorophenyl)methyl)piperazin-1-ylbut-2-enyloxy)acetic acid (SUN-1334H), cetirizine, fexofenadine, and terfenadine.

According to aspects of the present invention, the subject treated with metabolites or inhibitors thereof is not treated with an antibiotic.

The metabolites or inhibitors thereof may be provided per se or as part of a pharmaceutical composition.

Furthermore, the metabolites or metabolite inhibitors used for treating diseases (e.g. colitis) may be formulated in a single formulation (e.g. pharmaceutical composition) or may be provided in separate formulations (e.g. pharmaceutical composition).

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the metabolites or metabolite inhibitors of the present invention accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, intraperitoneal, intracardiac, intranasal, or intraocular injections.

In a particular embodiment, the metabolites and/or inhibitors thereof are formulated for rectal administration.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., the metabolite or inhibitor thereof) effective to prevent, alleviate, or ameliorate symptoms of the pathology or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.).

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

The timing of administration of the metabolites and metabolite inhibitors may take into account the natural rhythm of the microbial metabolome. Thus, for example if analysis shows that a particular metabolite of a microbe is at a peak in the morning hours and at a trough in the evening hours, then it may be recommended that a microbial composition which comprises this microbe, or metabolite itself is administered in the morning and not the evening so as not to alter the natural circadian rhythm of the microbiome. If the analysis of the rhythm of the microbiome shows that a particular metabolite of a microbe is at a peak in the morning hours and at a trough in the evening hours, then it may be recommended that a metabolite inhibitor which downregulates this metabolite is administered in the evening and not the morning so as not to alter the natural circadian rhythm of the microbial metabolome.

In order to analyze the rhythm of the microbiome metabolome, at least two samples, at least 3 samples, at least 4 samples, at least 5 samples, at least 6 samples or more of the microbiome metabolome should be measured during the course of a 24 hour period.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

It will be appreciated that the metabolites of the present invention may also be provided as part of a microbial composition. Such microbial compositions may be formulated in a food product, functional food or nutraceutical.

According to one embodiment, the microbial composition is not derived from fecal material.

According to still another embodiment, the microbial composition is devoid (or comprises only trace quantities) of fecal material (e.g, fiber).

The microbial composition may be in any suitable form, for example in a powdered dry form. In addition, the microbial composition may have undergone processing in order for it to increase its survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

It will be appreciated that as well as selecting a treatment according to the metabolites produced in the microbiome of the subject, the present inventors also propose use of the microbiome for monitoring the response of a subject to a treatment.

Thus, according to another aspect of the present invention there is provided a method of monitoring a treatment of a disease in a test subject:

(a) treating the test subject with at least one metabolite that is produced in microbiome of the subject;

(b) analyzing a signature of the microbiome of the test subject; and subsequently; and

(c) comparing the microbiome signature of the test subject with the microbiome signature of a healthy subject, wherein an increase in the similarity of the microbiome signature of the test subject with the microbiome signature of the healthy subject following the treating as compared to the similarity of the microbiome signature of the test subject with the microbiome signature of the healthy subject prior to the treating is indicative of an effective treatment.

In this aspect of the present invention, the subject is treated with at least one metabolite and subsequently his microbiome signature is analyzed.

Exemplary metabolites have been described herein above.

Microbial signatures comprise data points that are indicators of microbiome composition and/or activity. Thus, according to the present invention, changes in microbiomes can be detected and/or analyzed through detection of one or more features of microbial signatures.

In some embodiments, a microbial signature includes information relating to absolute amount of one or more types of microbes, and/or products thereof. In some embodiments, a microbial signature includes information relating to relative amounts of five, ten, twenty or more types of microbes and/or products thereof.

Examples of microbial products include, but are not limited to mRNAs, polypeptides, carbohydrates and metabolites.

In some embodiments, a microbial signature includes information relating to presence, level, and/or activity of at least ten types of microbes. In some embodiments, a microbial signature includes information relating to presence, level, and/or activity of between 5 and 100 types of microbes. In some embodiments, a microbial signature includes information relating to presence, level, and/or activity of between 100 and 1000 or more types of microbes. In some embodiments, a microbial signature includes information relating to presence, level, and/or activity of substantially all types of bacteria within the microbiome. In some embodiments, a microbial signature includes information relating to presence, level, and/or activity of substantially all types of microbes within the microbiome.

In some embodiments, a microbial signature includes information relating to presence, level, and/or activity of metabolites of at least ten types of microbes. In some embodiments, a microbial signature includes information relating to presence, level, and/or activity of metabolites of between 5 and 100 types of microbes. In some embodiments, a microbial signature includes information relating to presence, level, and/or activity of metabolites of between 100 and 1000 or more types of microbes. In some embodiments, a microbial signature includes information relating to presence, level, and/or activity of substantially metabolites of all types of bacteria within the microbiome. In some embodiments, a microbial signature includes information relating to presence, level, and/or activity of metabolites of substantially all types of microbes within the microbiome.

In some embodiments, a microbial signature includes information relating to presence, level, and/or activity of at least 10% of the metabolites of the microbes of the microbiome. In some embodiments, a microbial signature includes information relating to presence, level, and/or activity of at least 20% of the metabolites of the microbes of the microbiome. In some embodiments, a microbial signature includes information relating to presence, level, and/or activity of at least 30% of the metabolites of the microbes of the microbiome. In some embodiments, a microbial signature includes information relating to presence, level, and/or activity of at least 40% of the metabolites of the microbes of the microbiome. In some embodiments, a microbial signature includes information relating to presence, level, and/or activity of at least 50% of the metabolites of the microbes of the microbiome. In some embodiments, a microbial signature includes information relating to presence, level, and/or activity of at least 60% of the metabolites of the microbes of the microbiome. In some embodiments, a microbial signature includes information relating to presence, level, and/or activity of at least 70% of the metabolites of the microbes of the microbiome. In some embodiments, a microbial signature includes information relating to presence, level, and/or activity of at least 80% of the metabolites of the microbes of the microbiome. In some embodiments, a microbial signature includes information relating to presence, level, and/or activity of at least 90% of the metabolites of the microbes of the microbiome. In some embodiments, a microbial signature includes information relating to presence, level, and/or activity of 100% of the metabolites of the microbes of the microbiome.

According to this aspect of the present invention the microbiome signature includes a presence or level of at least one, at least 10, at least 20, at least 50, at least 100, at least 200, at least 300, at least 400, at least 500, at least 600, at least 700, at least 800, at least 900, at least 1000, at least 1200, at least 1500 or all the species of microbes of the microbiome.

In some embodiments, a microbiome signature comprises a level or set of levels of at least one, or at least five, or at least ten or more types of microbes (e.g. bacteria) or components or by-products thereof. In some embodiments, a microbial signature comprises a level or set of levels of at least one or at least five or at least ten or more DNA sequences. In some embodiments, a microbial signature comprises a level or set of levels of ten or more 16S rRNA gene sequences. In some embodiments, a microbial signature comprises a level or set of levels of 18S rRNA gene sequences. In some embodiments, a microbial signature comprises a level or set of levels of at least five or at least ten or more RNA transcripts. In some embodiments, a microbial signature comprises a level or set of levels of at least five or at least ten or more proteins. In some embodiments, a microbial signature comprises a level or set of levels of at least one or at least five or at least ten or more metabolites.

16S and 18S rRNA gene sequences encode small subunit components of prokaryotic and eukaryotic ribosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

Quantifying Microbial Levels:

In methods in accordance with the present invention, a microbial signature is obtained and/or determined by quantifying microbial levels. Methods of quantifying levels of microbes of various types are described herein below.

In some embodiments, determining a level or set of levels of one or more types of microbes or components or products thereof comprises determining a level or set of levels of one or more DNA sequences. In some embodiments, one or more DNA sequences comprise any DNA sequence that can be used to differentiate between different microbial types. In certain embodiments, one or more DNA sequences comprise 16S rRNA gene sequences. In certain embodiments, one or more DNA sequences comprise 18S rRNA gene sequences. In some embodiments, 1, 2, 3, 4, 5, 10, 15, 20, 25, 50, 100, 1,000, 5,000 or more sequences are amplified.

In some embodiments, a microbiota sample (e.g. fecal sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes or components or products thereof comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

In some embodiments, determining a level or set of levels of one or more types of microbes or components or products thereof comprises determining a level or set of levels of one or more microbial polypeptides. Methods of quantifying polypeptide levels are well known in the art and include but are not limited to Western analysis and mass spectrometry. These and all other basic polypeptide detection procedures are described in Ausebel et al. infra.

In some embodiments, determining a level or set of levels of one or more types of microbes or components or products thereof comprises determining a level or set of levels of one or more microbial metabolites. Methods of determining microbial metabolites are described herein above.

According to one embodiment of this aspect of the present invention two microbiome signatures can behave a statistically significant similar signature when they comprise at least 50% of the same microbes, at least 60% of the same microbes, at least 70% of the same microbes, at least 80% of the same microbes, at least 90% of the same microbes, at least 91% of the same microbes, at least 92% of the same microbes, at least 93% of the same microbes, at least 94% of the same microbes, at least 95% of the same microbes, at least 96% of the same microbes, at least 97% of the same microbes, at least 98% of the same microbes, at least 99% of the same microbes or 100% of the same microbes.

Additionally, or alternatively, microbiomes may have a statistically significant similar signature when the quantity (e.g. occurrence) in the microbiome of at least one microbe of interest is identical. According to another embodiment, microbiomes may have a statistically significant similar signature when the relative ratio in the microbiome of at least 10% of its microbes are identical. According to another embodiment, microbiomes may have a statistically significant similar signature when the relative ratio in the microbiome of at least 20% of its microbes are identical. According to another embodiment, microbiomes may have a statistically significant similar signature when the relative ratio in the microbiome of at least 30% of its microbes are identical. According to another embodiment, microbiomes may have a statistically significant similar signature when the relative ratio in the microbiome of at least 40% of its microbes are identical. According to another embodiment, microbiomes may have a statistically significant similar signature when the relative ratio in the microbiome of at least 50% of its microbes are identical. According to another embodiment, microbiomes may have a statistically significant similar signature when the relative ratio in the microbiome of at least 60% of its microbes are identical. According to another embodiment, microbiomes may have a statistically significant similar signature when the relative ratio in the microbiome of at least 70% of its microbes are identical. According to another embodiment, microbiomes may have a statistically significant similar signature when the relative ratio in the microbiome of at least 80% of its microbes are identical. According to another embodiment, microbiomes may have a statistically significant similar signature when the relative ratio in the microbiome of at least 90% of its microbes are identical. Thus, the fractional percentage of microbes (e.g. relative amount, ratio, distribution, frequency, percentage, etc.) of the total may be statistically similar.

Additionally, or alternatively, microbiomes may have a statistically significant similar signature when the quantity (e.g. occurrence) in the microbiome of at least one metabolite of interest is identical. According to another embodiment, microbiomes may have a statistically significant similar signature when the relative ratio in the microbiome of at least 10% of its metabolites are identical. According to another embodiment, microbiomes may have a statistically significant similar signature when the relative ratio in the microbiome of at least 20% of its metabolites are identical. According to another embodiment, microbiomes may have a statistically significant similar signature when the relative ratio in the microbiome of at least 30% of its metabolites are identical. According to another embodiment, microbiomes may have a statistically significant similar signature when the relative ratio in the microbiome of at least 40% of its metabolites are identical. According to another embodiment, microbiomes may have a statistically significant similar signature when the relative ratio in the microbiome of at least 50% of its metabolites are identical. According to another embodiment, microbiomes may have a statistically significant similar signature when the relative ratio in the microbiome of at least 60% of its metabolites are identical. According to another embodiment, microbiomes may have a statistically significant similar signature when the relative ratio in the microbiome of at least 70% of its metabolites are identical. According to another embodiment, microbiomes may have a statistically significant similar signature when the relative ratio in the microbiome of at least 80% of its metabolites are identical. According to another embodiment, microbiomes may have a statistically significant similar signature when the relative ratio in the microbiome of at least 90% of its metabolites are identical. Thus, the fractional percentage of metabolites (e.g. relative amount, ratio, distribution, frequency, percentage, etc.) of the total may be statistically similar.

According to another embodiment, in order to classify a microbe as belonging to a particular genus, family, order, class or phylum, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular genus. According to a particular embodiment, the sequence homology is at least 95%.

According to another embodiment, in order to classify a microbe as belonging to a particular species, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular species. According to a particular embodiment, the sequence homology is at least 97%.

In determining whether a nucleic acid or protein is substantially homologous or shares a certain percentage of sequence identity with a sequence of the invention, sequence similarity may be defined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www(dot)ncbi(dot)nlm(dot)nih(dot)gov for more details.

According to still another embodiment, two microbiome signatures can be classified as being similar, if the relative number of genes belonging to a particular pathway is similar.

According to still another embodiment, two microbiome signatures can be classified as being similar, if the relative amount of a product generated by the microbes is similar.

Any of the analytical methods described herein can be embodied in many forms. For example, it can be embodied in on a tangible medium such as a computer for performing the method operations. It can be embodied on a computer readable medium, comprising computer readable instructions for carrying out the method operations. It can also be embodied in electronic device having digital computer capabilities arranged to run the computer program on the tangible medium or execute the instruction on a computer readable medium.

Computer programs implementing the analytical method of the present embodiments can commonly be distributed to users on a distribution medium such as, but not limited to, CD-ROMs or flash memory media. From the distribution medium, the computer programs can be copied to a hard disk or a similar intermediate storage medium. In some embodiments of the present invention, computer programs implementing the method of the present embodiments can be distributed to users by allowing the user to download the programs from a remote location, via a communication network, e.g., the internet. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

Agents which may be used for analyzing the similarity of a microbiome signature of a test subject with a microbiome signature of a healthy subject may include a primer or set of primers for amplifying 16S rRNA or 18S rRNA. Such agents may be provided in a kit for monitoring a treatment of a disease in a test subject. The kit of this embodiment may comprise additional reagents required for subsequent sequencing reactions.

In the case of a gene (DNA) or RNA, the agent may be an oligonucleotide which hybridizes specifically to the DNA or RNA of interest.

The oligonucleotide may be in the form of an amplification primer. In this case, the kit may comprise additional components to perform an amplification reaction such as enzymes, salts and buffers.

Alternatively, the oligonucleotide may be attached to a solid surface (i.e. array). Several substrates suitable for the construction of arrays are known in the art, and one skilled in the art will appreciate that other substrates may become available as the art progresses. The substrate may be a material that may be modified to contain discrete individual sites appropriate for the attachment or association of the oligonucleotide and is amenable to at least one detection method. Non-limiting examples of substrate materials include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. In an exemplary embodiment, the substrates may allow optical detection without appreciably fluorescing.

A substrate may be planar, a substrate may be a well, i.e. a 364 well plate, or alternatively, a substrate may be a bead. Additionally, the substrate may be the inner surface of a tube for flow-through sample analysis to minimize sample volume. Similarly, the substrate may be flexible, such as a flexible foam, including closed cell foams made of particular plastics.

The oligonucleotide or oligonucleotides may be attached to the substrate in a wide variety of ways, as will be appreciated by those in the art. The oligonucleotide may either be synthesized first, with subsequent attachment to the substrate, or may be directly synthesized on the substrate. The substrate and the oligonucleotide may be derivatized with chemical functional groups for subsequent attachment of the two. For example, the substrate may be derivatized with a chemical functional group including, but not limited to, amino groups, carboxyl groups, oxo groups or thiol groups. Using these functional groups, the oligonucleotide may be attached using functional groups on the oligonucleotide either directly or indirectly using linkers.

The oligonucleotide may also be attached to the substrate non-covalently. For example, a biotinylated oligonucleotide can be prepared, which may bind to surfaces covalently coated with streptavidin, resulting in attachment. Alternatively, a oligonucleotide or oligonucleotides may be synthesized on the surface using techniques such as photopolymerization and photolithography. Additional methods of attaching oligonucleotides to arrays and methods of synthesizing oligonucleotides on substrates are well known in the art, i.e. VLSIPS technology from Affymetrix (e.g., see U.S. Pat. No. 6,566,495, and Rockett and Dix, “DNA arrays: technology, options and toxicological applications,” Xenobiotica 30(2):155-177, all of which are hereby incorporated by reference in their entirety).

In one embodiment, the oligonucleotide or oligonucleotides attached to the substrate are located at a spatially defined address of the array. Arrays may comprise from about 1 to about several hundred thousand addresses or more. In one embodiment, the array may be comprised of less than 10,000 addresses. In another alternative embodiment, the array may be comprised of at least 10,000 addresses. In yet another alternative embodiment, the array may be comprised of less than 5,000 addresses. In still another alternative embodiment, the array may be comprised of at least 5,000 addresses. In a further embodiment, the array may be comprised of less than 500 addresses. In yet a further embodiment, the array may be comprised of at least 500 addresses.

An oligonucleotide may be represented more than once on a given array. In other words, more than one address of an array may be comprised of the same oligonucleotide. In some embodiments, two, three, or more than three addresses of the array may be comprised of the same oligonucleotide. In certain embodiments, the array may comprise control oligonucleotides and/or control addresses. The controls may be internal controls, positive controls, negative controls, or background controls.

In one embodiment, the array may comprise an agent which can quantify or qualify the presence of a metabolite enriched in a host microbiome (subject being treated) compared to its level in a healthy microbiome. In another embodiment, the array may comprise an agent which can quantify or qualify the presence of a metabolite depleted in a host microbiome (subject being treated) compared to its level in a healthy microbiome. In yet another embodiment, the array may comprise an agent which can quantify or qualify the presence of a metabolite up-regulated in a host microbiome (subject being treated) compared to its level in a healthy microbiome. In still another embodiment, the array may comprise an agent which can quantify or qualify the presence a metabolite down-regulated in a host microbiome (subject being treated) compared to its level in a healthy microbiome. In still yet another embodiment, the array may comprise an agent which can quantify or qualify the presence of a metabolite degraded in the host microbiome (subject being treated) compared to its level in a healthy microbiome. In an alternative embodiment, the array may comprise an agent which can quantify or qualify the presence of a metabolite stabilized in the host microbiome (subject being treated) compared to its level in a healthy microbiome.

The present invention also contemplates analyzing metabolites of the microbiome in order to diagnose disease.

Thus, according to yet another aspect of the present invention there is provided a method of diagnosing an inflammatory bowel disease (e.g. colitis) in a subject comprising analyzing the amount of a metabolite selected from the group consisting of taurine, histamine, putrescine and spermine produced in the microbiome of the subject, when a decrease in taurine below a predetermined level and/or an increase in histamine or spermine above a predetermined level is indicative of the inflammatory bowel disease (e.g. colitis).

The present invention contemplates analyzing the amount of only one of the metabolites listed herein above, two of the metabolites listed herein above, three of the metabolites listed herein above or all of the metabolites listed herein above. Preferably, at least one of the metabolites analyzed is taurine.

As used herein, the term “diagnosing” refers to classifying a disease, a condition or a symptom, or to determining a severity of the disease, condition or symptom monitoring disease progression, forecasting an outcome of a disease and/or prospects of recovery.

The predetermined level may be ascertained by analyzing levels of the metabolites in samples of subjects known to have colitis or known to be healthy. An upregulation of taurine at least 1.5 fold, 2 fold, 3 fold, 4 fold, or 5 fold as compared to the level of taurine in a sample from a healthy subject is indicative that the subject has colitis. Conversely, a downregulation of histamine, spermine or putrescine by at least 1.5 fold, 2 fold, 3 fold, 4 fold, or 5 fold as compared to the level of taurine in a sample from a healthy subject is indicative that the subject has colitis.

As mentioned, microbial signatures comprise may be used as indicators of microbiome composition and/or activity.

The present inventors further propose that the microbial signature of a microbiome of a diseased subject (also referred to herein as a pathological microbiome) may be used to determine the therapeutic effect of an agent.

Thus according to still another aspect of the present invention there is provided a method of determining the therapeutic effect of an agent:

(a) exposing a pathological microbiome to the agent; and

(b) comparing the signature of the pathological microbiome following the exposing with a reference signature of a healthy microbiome, wherein when the signature of the microbiome is statistically significantly similar to the healthy microbiome reference signature, it is indicative that the agent has a therapeutic effect on the microbiome.

As used herein, the phrase “pathological microbiome” refers to a microbiome derived from a subject who is known to have a disease (e.g. metabolic disease such as diabetes, or pre-diabetes, cancer).

Exposure of the pathological microbiome to the agent may be effected ex vivo or in vivo.

Microbiome signatures and methods of analyzing same have been described herein above.

Microbial signatures comprise data points that are indicators of microbiome composition and/or activity. Thus, according to the present invention, changes in microbiomes can be detected and/or analyzed through detection of one or more features of microbial signatures.

According to a particular embodiment, the signature is a metabolic signature.

Agents which may be analyzed according to this aspect of the present invention include inorganic or organic compounds; small molecules (i.e., less than 1000 Daltons) or large molecules (i.e., above 1000 Daltons); biomolecules (e.g. proteinaceous molecules, including, but not limited to, peptide, polypeptide, post-translationally modified protein, antibodies etc.) or a nucleic acid molecule (e.g. double-stranded DNA, single-stranded DNA, double-stranded RNA, single-stranded RNA, or triple helix nucleic acid molecules) or chemicals. Therapeutic agents may be natural products derived from any known organism (including, but not limited to, animals, plants, bacteria, fungi, protista, or viruses) or from a library of synthetic molecules. Therapeutic agents can be monomeric as well as polymeric compounds.

According to a particular embodiment, the agent is a metabolite, as further described herein above.

According to another embodiment, the metabolite is one that alters the composition or function of the microbiome.

Methods of analyzing similarity or differences in microbial signatures are described herein above.

It will be appreciated that the method of this aspect of the present invention may be extended by:

(i) analyzing the microbiome signature of the subject prior to step (a); and/or

(ii) providing the agent to a subject having a pathological microbiome (i.e. diseased subject) once it has been verified that the agent is therapeutic.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture”

Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Mice:

C57Bl/6 mice were purchased from Harlan and allowed to acclimatize to the animal facility environment for 2 weeks before used for experimentation. Germ-free Swiss Webster mice were born in the Weizmann Institute germ-free facility and routinely monitored for sterility.

Asc^{-/-} mice (Sutterwala et al., 2006), Casp1^{-/-} mice (Kuida et al., 1995), Nlrp6^{-/-} mice (Elinav et al., 2011), IL-18^{-/-} mice (Takeda et al., 1998), and Nlrp3^{-/-} mice (Mariathasan et al., 2006), were previously described. In all experiments, age- and gender-matched mice were used. Mice were 8-9 weeks of age at the beginning of experiments. For cohousing experiments, age- and gender-matched wild-type germ-free and knockout mice were cohoused in new cages at 1:1 ratios for 4 weeks. For experiments involving cohousing, only female mice were used.

For bone marrow chimera experiments, mice were given a sublethal dose of total body irradiation (900 Gy). 16 hours later, mice were transplanted with 3×10⁶ bone marrow cells. Mice were analyzed 8 weeks following reconstitution.

For IL-18 rescue experiments, recombinant IL-18 (MBL, Cat#B004-2) was injected intraperitoneally at a concentration of 1 μg per mouse for 5 days, and not injected for 2 days prior to DSS treatment. IL-18 injection to Swiss Webster germ-free mice and Nlrp6^{-/-} mice was performed twice a day for 3 days, while the mice were kept sterile using a cage-autonomous system (Hecht et al., 2014).

Fresh stool samples from mice were collected in tubes, immediately frozen in liquid nitrogen upon collection, and stored at −80° C. until DNA isolation. For antibiotic treatment, mice were given a combination of vancomycin (1 g/l), ampicillin (1 g/l), kanamycin (1 g/l), and metronidazole (1 g/l) in their drinking water (Fagarasan et al., 2002; Ichinohe et al., 2011; Rakoff-Nahoum et al., 2004). All antibiotics were obtained from Sigma Aldrich.

All experimental procedures were approved by the local IACUC.

DSS Colitis:

Mice were treated with 1.5% (w/v) DSS (M.W.=36,000-50,000 Da; MP Biomedicals) in their drinking water for 7 days followed by regular access to water. The animals were weighed daily and monitored for signs of distress as well as rectal bleeding.

For DSS rescue experiments, mice were receiving taurine in the drinking water prior to DSS treatment in a concentration of 100 mg/ml for 14 days.

Colonoscopy:

Colonoscopy was performed using a high-resolution mouse video endoscopic system (Carl Storz, Tuttlingen, Germany). The severity of colitis was blindly scored using MEICS (Murine Endoscopic Index of Colitis Severity), which is based on five parameters: granularity of mucosal surface; vascular pattern; translucency of the colon mucosa; visible fibrin; and stool consistency (Becker et al., 2006).

Measuring Colonic Epithelial Barrier Permeability by FITC-Dextran:

On the day of the assay, 4 kDa fluorescein isothiocyanate (FITC)-dextran was dissolved in phosphate buffered saline (PBS) to a concentration of 80 mg ml⁻¹. Mice were fasted for 4 hours prior to gavage with 150 μl dextran. Mice were anesthetized 3 hours following gavage and blood was collected, centrifuged at 1,000×g for 12 min at 4° C. Serum was collected and fluorescence was quantified at an excitation wavelength of 485 nm and 535 nm emission wavelength.

16S qPCR Protocol for Quantification of Bacterial DNA:

DNA was extracted from liver samples (using MoBio PowerSoil kit). DNA concentration was calculated using a standard curve of known DNA concentrations from E. coli K12. 16S qPCR using primers identifying different regions of the V6 16S gene was performed using Kappa SYBR fast mix.

Absolute number of bacteria in the samples was then approximated as DNA amount in a sample/DNA molecule mass of bacteria.

Western Blot Analysis:

Colons were excised and washed thoroughly with PBS, homogenized in RIPA buffer containing protease inhibitors, incubated for 20 min in 4° C. and centrifuged for 20 min, 14,000 rpm at 4° C. Samples were separated on 15% acrylamide gels and transferred onto nitrocellulose membranes. Western blot analysis was performed using anti-caspase 1 p20 polyclonal antibody (Adipogen, AG-20B-0042B-C100) and Goat anti mouse antibody (Jackson ImmunoResearch, 115-035-003). Band density was calculated using ImageJ software.

Colonic Explants:

Colon pieces, 0.5 cm long, from the proximal colon were removed, rinsed with PBS, and weighed. The tissue explants were cultured for 24 hrs in DMEM medium containing 10% FBS, L-glutamine, penicillin, and streptomycin at 37° C. Culture medium was collected, centrifuged and the resulting supernatant stored in aliquots at −20° C.

ELISA:

Concentrations of IL-18 in the serum or culture supernatants were measured using ELISA. Plates were coated over night with IL-18 (MBL, B004-2), incubated for 4 hours with supernatant or serum. Samples were then washed and plates were incubated with anti-mouse IL-18-biotin antibody (D048-6 MBL) for 1 hour and HRP-Avidin (BioLegend) for 1 hour before quantification.

In-Vitro Assays:

Colon explants were incubated with 70 mM of taurine (Sigma Aldrich, T0625), histamine (Sigma Aldrich, H7125) or spermine (Sigma Aldrich, S3256) for 24 hours. For AMP induction in-vitro, 3 μg IL-18 were added to colon explants for 7 hours, then the explants were maintained in RNAlater and RNA was extracted.

Ang4 In-Vitro Culture:

Murine Ang4 was expressed in E. coli and purified as previously described (Holloway et al, 2001). Stool pellets were collected and cultured in anaerobic conditions for 8 hours with rAng4, centrifuged and further analyzed using 16S rRNA sequencing.

Gene Expression Analysis:

Tissues were preserved in RNAlater solution (Ambion) and subsequently homogenized in Tri Reagent (Fox et al.). RNA was purified according to the manufacturer's instructions. Two microgram of total RNA was used to generate cDNA (HighCapacity cDNA Reverse Transcription kit; Applied Biosystems). RealTime-PCR was performed using gene-specific primer/probe sets (Applied Biosystems) and Kapa Probe Fast qPCR kit (Kapa Biosystems) on a Viia7 instrument (Applied Biosystems). PCR conditions were 95° C. for 20 s, followed by 40 cycles of 95° C. for 3 s and 60° C. for 30 s. Data were analyzed using the deltaCt method with hprt serving as the reference housekeeping gene.

Global RNA Sequencing:

Colon tissues were preserved in RNAlater solution (Ambion) and homogenized in Trizol reagent (Invitrogen). RNA was extracted using chloroform, precipitated with isopropanol, and subsequently washed with 70% ethanol. 400 ng of total RNA were used for library preparation. mRNA was heat fragmented, captured with 12.5 μl of Dynabeads oligo(dT) (Life technologies), and washed according to the manufacturer's instructions. Purified messenger RNA was eluted at 65° C. with 7.8 μl of 10 mM Tris-Cl pH 7.5. For indexed cDNA preparation, samples were incubated at 72° C. for 3 min and immediately transferred to 4° C., followed by RT reaction with the mix containing 10 mM DTT, 4 mM dNTP, 2.5 U/μl Superscript III RT enzyme in 50 mM Tris-HCl (pH 8.3), and 3 mM MgCl2. The reaction was performed in a thermo cycler (Eppendorf): 60 min at 42° C., and 15 min at 70° C. Indexed samples with equivalent amount of cDNA were pooled, RNAse treated, and the product was purified with 0.9× volumes of SPRI beads. The library was completed and amplified through a 12-cycle PCR reaction with 0.5 μM of P5_Rd1 and P7_Rd2 primers and PCR ready mix (Kapa Biosystems). The forward primer contains the Illumina P5-Read1 sequences and the reverse primer contains the P7-Read2 sequences. The amplified pooled library was purified with 0.8× volumes of SPRI beads to remove primer leftovers. Library concentration was measured using Qubit fluorometer (Life Technologies) and mean molecule size was determined with a 2200 TapeStation instrument (Agilent). Libraries were sequenced using an Illumina HiSeq 1500. Reads were analyzed as previously described (Lavin et al., 2014).

Functional assignment of reads was analyzed. RNA seq values were capped at the value of one read and normalized per each sample. Presented are heat-maps following Hierarchical clustering colored in a relative color scheme. Functional enrichment was tested for GO annotated components enriched with genes whose fold change is greater than 2 or smaller than 0.5 in IL-18^{-/-} compared to WT. Displayed are FDR corrected Hypergeometric p-values for enrichment.

Taxonomic Microbiota Analysis:

Frozen fecal samples were processed for DNA isolation using the MoBio PowerSoil kit according to the manufacturer's instructions. For the 16S rRNA gene PCR amplification, 1 ng of the purified fecal DNA was used for PCR amplification. Amplicons spanning the variable region 1/2 (V1/2) of the 16S rRNA gene were generated by using the following barcoded primers: Fwd 5′-XXXXXXXXAGAGTTTGATCCTGGCTCAG-3′ (SEQ ID NO: 1), Rev 5′-TGCTGCCTCCCGTAGGAGT-3′ (SEQ ID NO: 2), where X represents a barcode base. The reactions were subsequently pooled and cleaned (PCR clean kit, Promega), and the PCR products were then sequenced on an Illumina MiSeq in 500 bp paired-end method. The reads were then processed using the QIIME (Quantitative Insights Into Microbial Ecology, www(dot)qiime(dot)org) analysis pipeline as described (Caporaso et al., 2010; Elinav et al., 2011). In brief, fasta quality files and a mapping file indicating the barcode sequence corresponding to each sample were used as inputs, reads were split by samples according to the barcode, taxonomical classification was performed using the RDP-classifier, and an OTU table was created. Closed-reference OTU mapping was employed using the Greengenes database. Rarefaction was used to exclude samples with insufficient count of reads per sample. Sequences sharing 97% nucleotide sequence identity in the V2 region were binned into operational taxonomic units (97% ID OTUs). For beta-diversity, weighted unifrac measurements were plotted according to the two principal coordinates based on 1,000 reads per sample. The OTU tables used for our analyses are made accessible online.

Metagenomic Sequence Mapping:

Illumina sequencing reads were mapped to a gut microbial gene catalog (Qin et al., 2010) using GEM mapper (Aaron et al., 2008) with the following parameters:

-m 0:08 - s 0 -q offset33- gemqualitythreshold 26.

Functional Assignment:

Reads mapped to the gut microbial gene catalog were assigned a KEGG (Kanehisa and Goto, 2000; Kanehisa et al., 2014). Genes were subsequently mapped to KEGG modules and pathways. For the KEGG pathway analysis, only pathways whose gene coverage was above 0.2 were included. Bacterial assignment to metabolic pathways was done by mapping of metagenomic reads to genes from the Histidine metabolism (ko00330) pathway. Mapped reads were extracted and re-mapped to a bacterial genomes database. Reads that were successfully mapped were grouped into genera, and those not mapped were marked as ‘unknowns’.

Ang4 Protein Analysis by Mass Spectrometry:

Samples were subjected to in-solution tryptic digestion following buffer exchange. All chemicals are from Sigma Aldrich, unless stated otherwise. Feces samples were dissolved in cold PBS, centrifuged, and supernatant was filtered through a 0.45 um filter. Fitrate was loaded onto 10 kDa molecular weight cutoff filter and centrifuged. 200 μl of 8M urea, 0.1M Tris pH 8.0, 5 mM DTT were added to the filter unit, incubated at room temp for 20 min and centrifuged at 14,000×g for 20 min. A second wash with the same buffer was performed. 100 μl of 8M urea, 0.1M Tris pH 8.0, 10 mM iodoacetamide were then added and incubated in the dark for 30 min at 21° C. Proteins were washed twice with 100 ul 2M urea in 100 mM Tris pH 7.6 and were then digested with trypsin overnight at 37° C. Digested proteins were then centrifuged, acidified with trifloroacetic acid, desalted using solid-phase extraction columns (Oasis H L B, Waters, Milford, Mass., USA), and stored in −80° C. until analysis.

Liquid Chromatography—

ULC/MS grade solvents were used for all chromatographic steps. Each sample was loaded using split-less nano-Ultra Performance Liquid Chromatography (10 kpsi nanoAcquity; Waters, Milford, Mass., USA). The mobile phase was: A) H₂O+0.1% formic acid and B) acetonitrile+0.1% formic acid. Desalting of the samples was performed online using a reversed-phase C18 trapping column (180 μm internal diameter, 20 mm length, 5 μm particle size; Waters). The peptides were then separated using a T3 HSS nano-column (75 μm internal diameter, 250 mm length, 1.8 μm particle size; Waters) at 0.35 μL/min. Peptides were eluted from the column into the mass spectrometer using the following gradient: 4% to 35% B in 105 min, 35% to 90% B in 5 min, maintained at 95% for 5 min and then back to initial conditions.

Mass Spectrometry:

The nanoUPLC was coupled online through a nanoESI emitter (10 μm tip; New Objective; Woburn, Mass., USA) to a quadrupole orbitrap mass spectrometer (Q Exactive Plus, Thermo Scientific) using a FlexIon nanospray apparatus (Proxeon).

Data was acquired in parallel reaction monitoring (PRM) mode, where MS2 resolution was set to 35,000 and the maximum injection time set to 200 ms. Inclusion list of peptides to be monitored included Ang4 peptides.

Data Analysis:

Raw data was loaded into Skyline (https://skyline(dot)gs(dot)washington(dot)edu/), peak boundaries and relevant transitions were manually refined, and peak intensities were calculated.

Metabolomics Study:

Fecal samples were collected, immediately frozen in liquid nitrogen and stored at −80° C. Sample preparation and analysis was performed by Metabolon Inc. Samples were prepared using the automated MicroLab STAR™ system from (Hamilton). To remove protein, dissociate small molecules bound to protein or trapped in the precipitated protein matrix, and to recover chemically diverse metabolites, proteins were precipitated with methanol. The resulting extract was divided into five fractions: one for analysis by UPLC-MS/MS with positive ion mode electrospray ionization, one for analysis by UPLC-MS/MS with negative ion mode electrospray ionization, one for LC polar platform, one for analysis by GC-MS, and one sample was reserved for backup. Samples were placed briefly on a TurboVap® (Zymark) to remove the organic solvent. For LC, the samples were stored overnight under nitrogen before preparation for analysis. For GC, each sample was dried under vacuum overnight before preparation for analysis.

Data Extraction and Compound Identification:

Raw data was extracted, peak-identified and QC processed using Metabolon's hardware and software. Compounds were identified by comparison to library entries of purified standards or recurrent unknown entities. Metabolite Quantification and Data Normalization: Peaks were quantified using area-under-the-curve. For studies spanning multiple days, a data normalization step was performed to correct variation resulting from instrument inter-day tuning differences. Statistical analysis was done by Welch's two-sample t-test. For statistical significance testing, p-values are given and q-values for the level of 0.05 is the false positive rate.

Statistical Analysis:

Data are expressed as mean±SEM. P-values <0.05 were considered significant. * p<0.05; ** p<0.01; *** p<0.001; **** p<0.0001. Pairwise comparison was performed using Student's t test. Comparison between multiple groups was performed using ANOVA, and Mann-Whitney U-test was used to correct for multiple comparisons.

### Results

### The Microbiota Modulates its Own Niche by Activation of the NLRP6 Inflammasome and Downstream Anti-Microbial Peptide Secretion

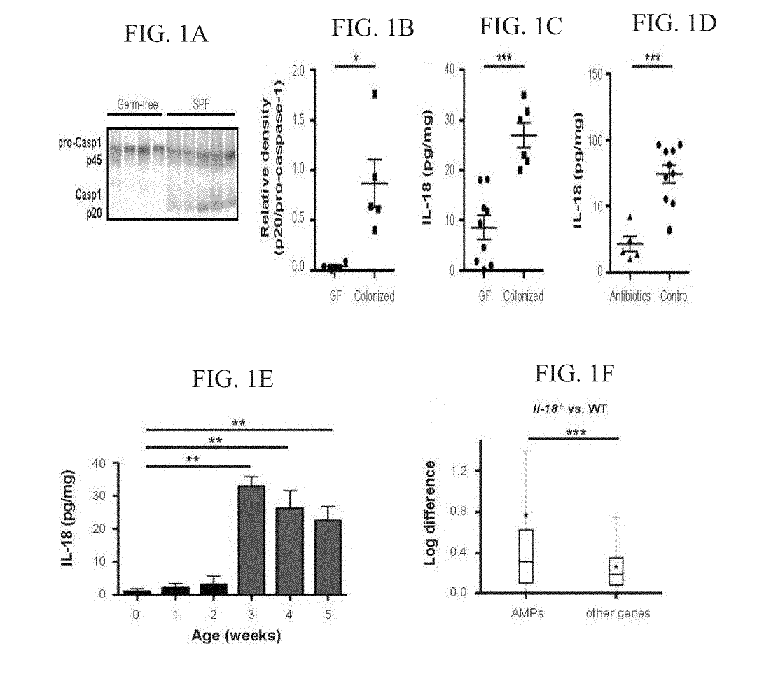

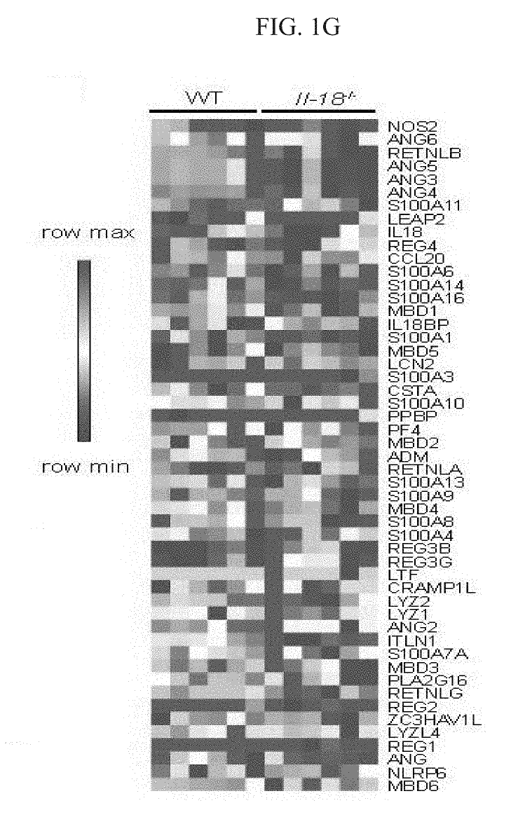

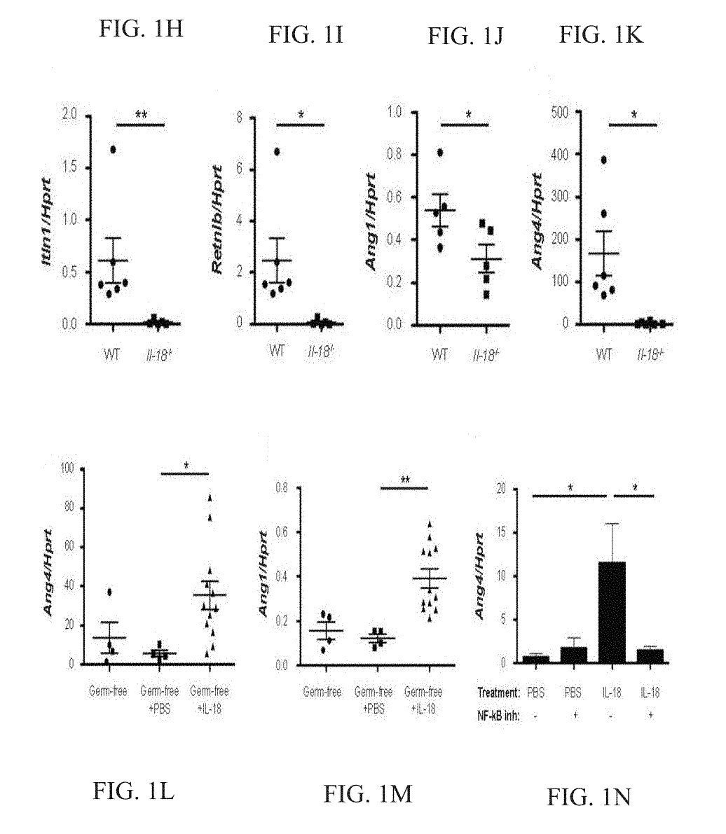

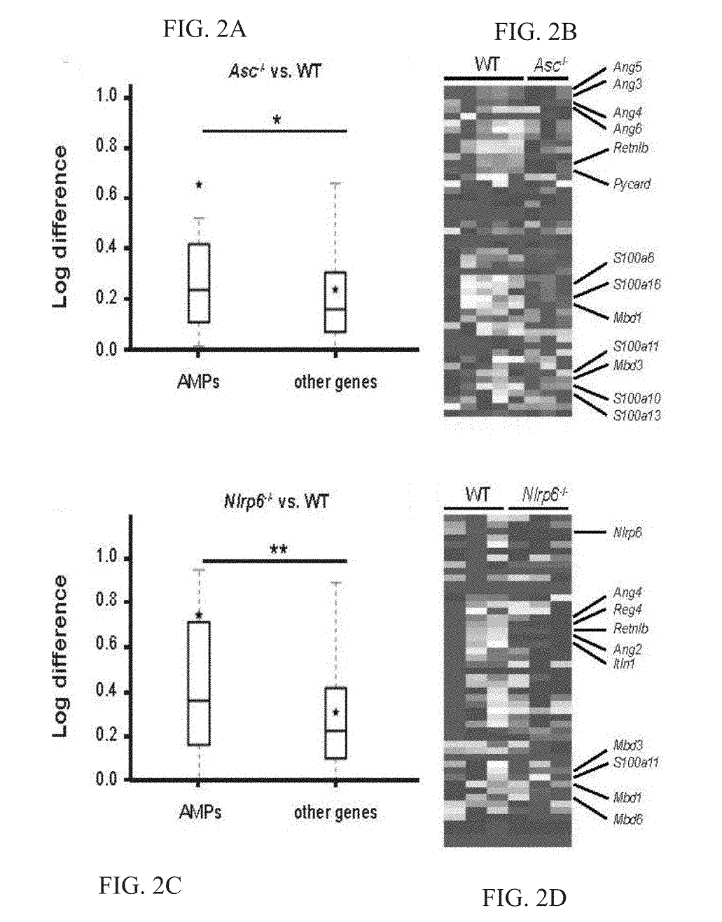

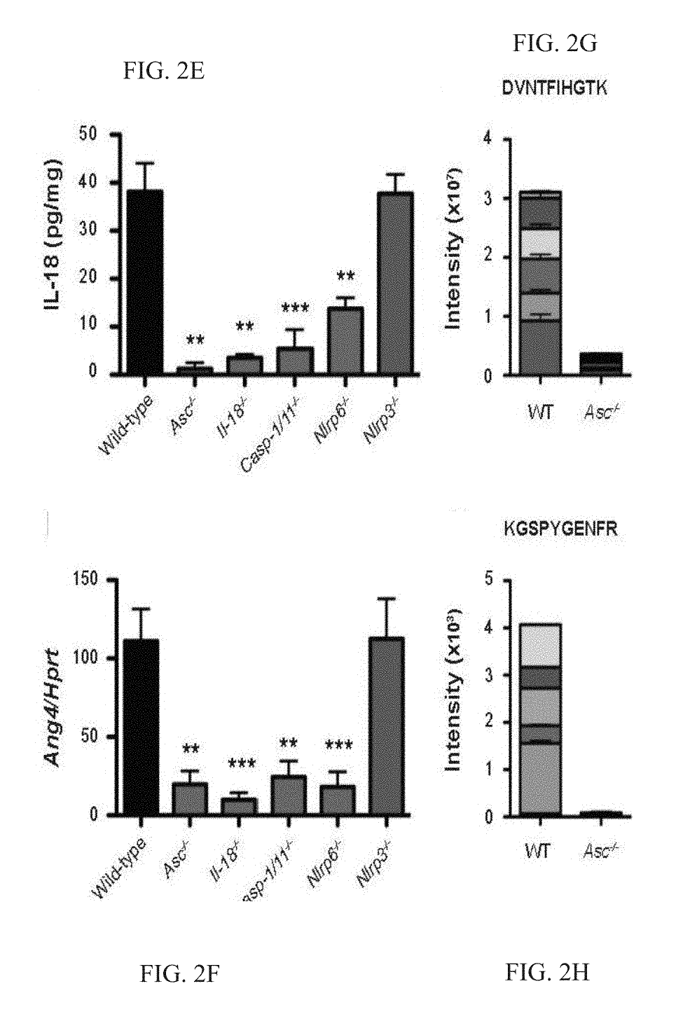

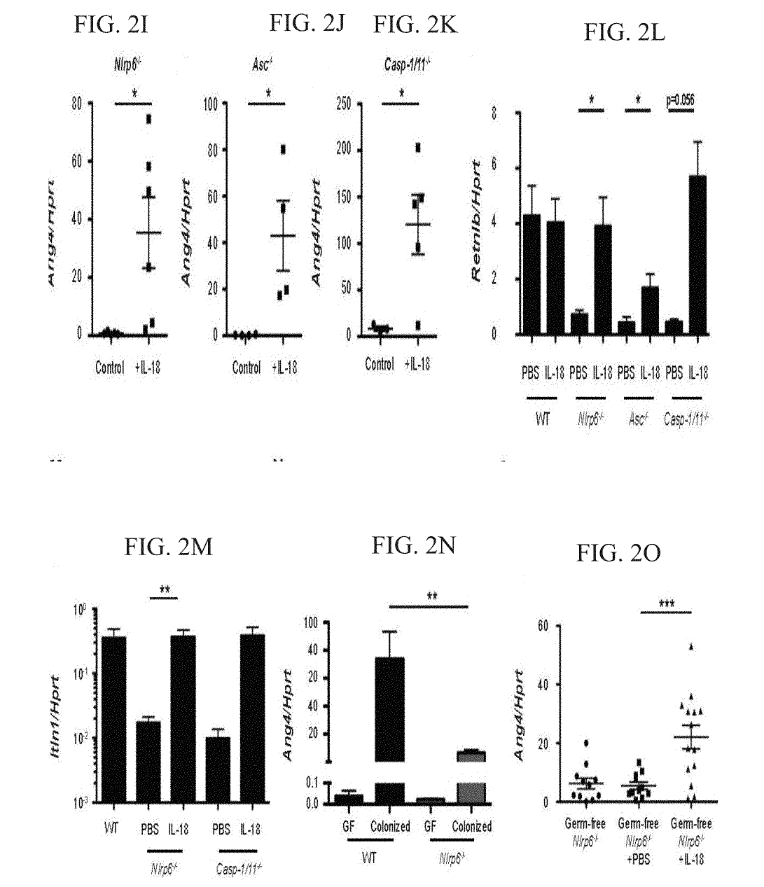

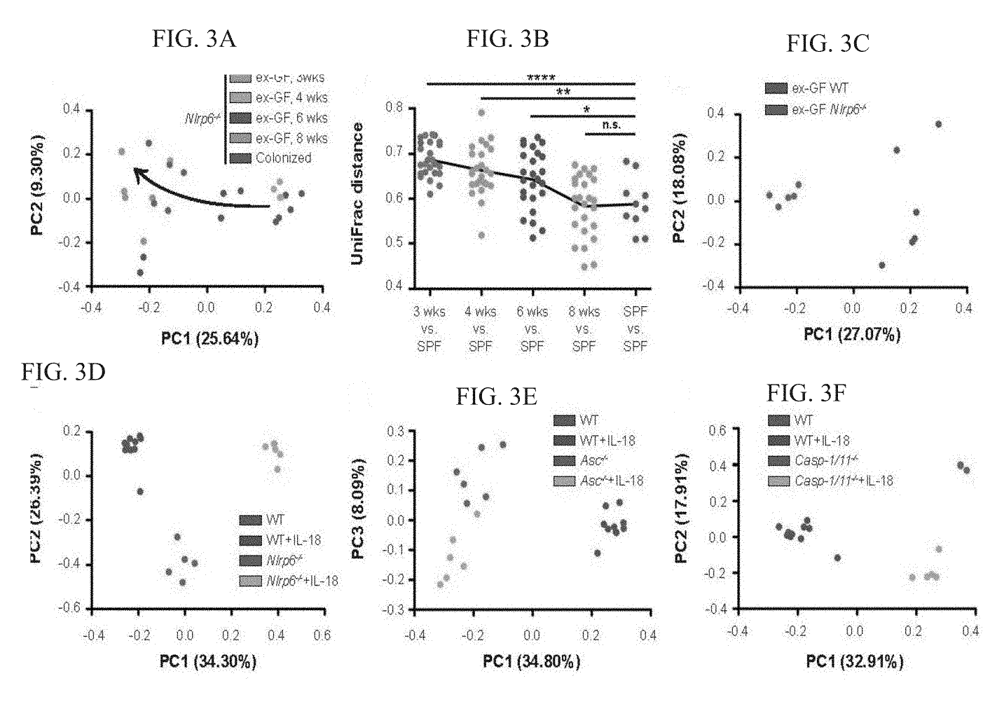

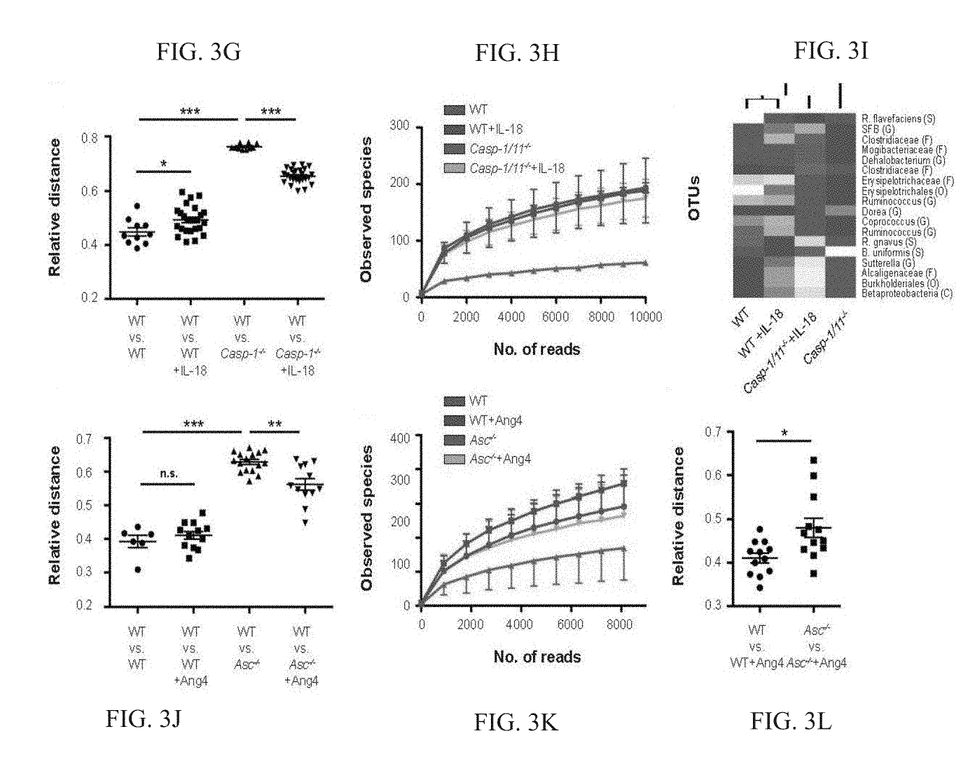

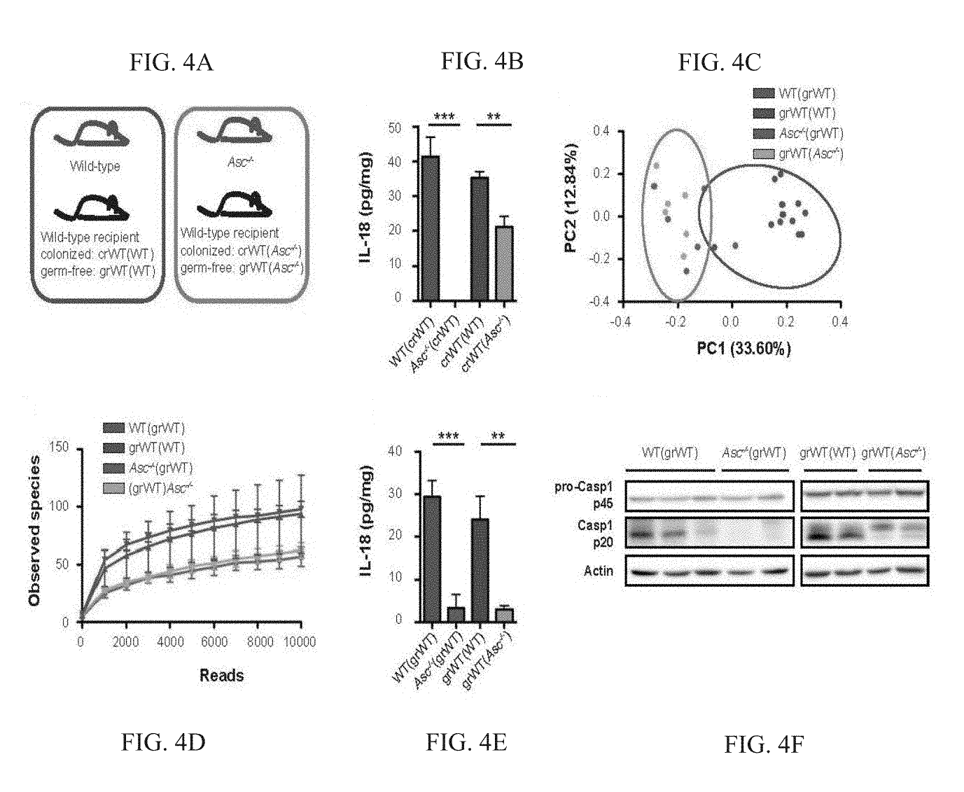

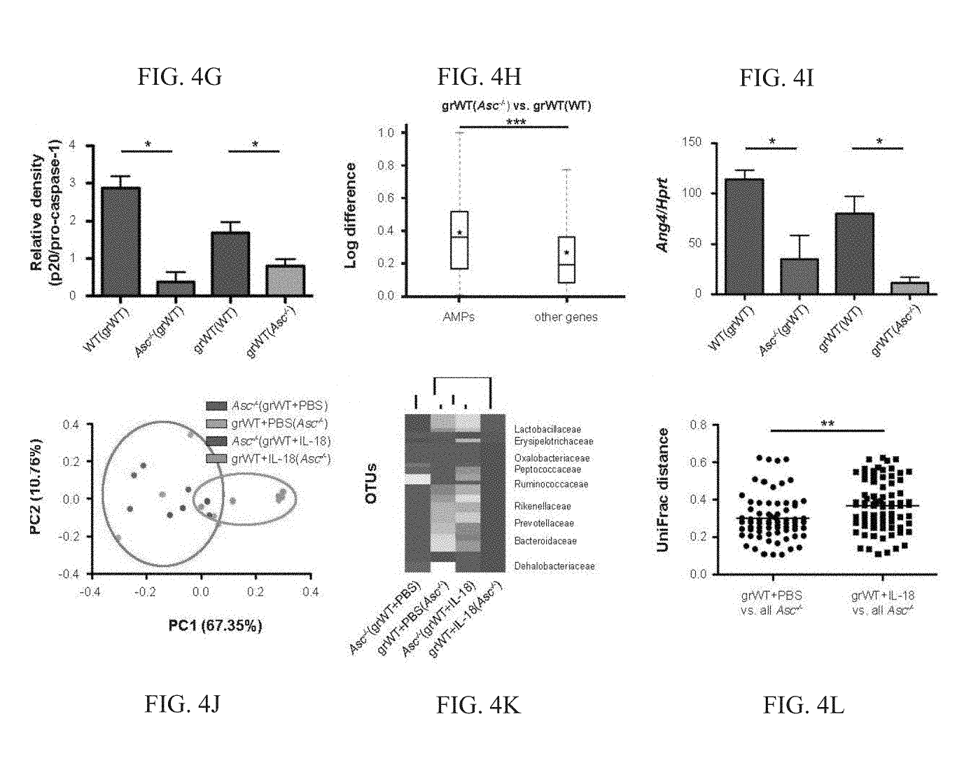

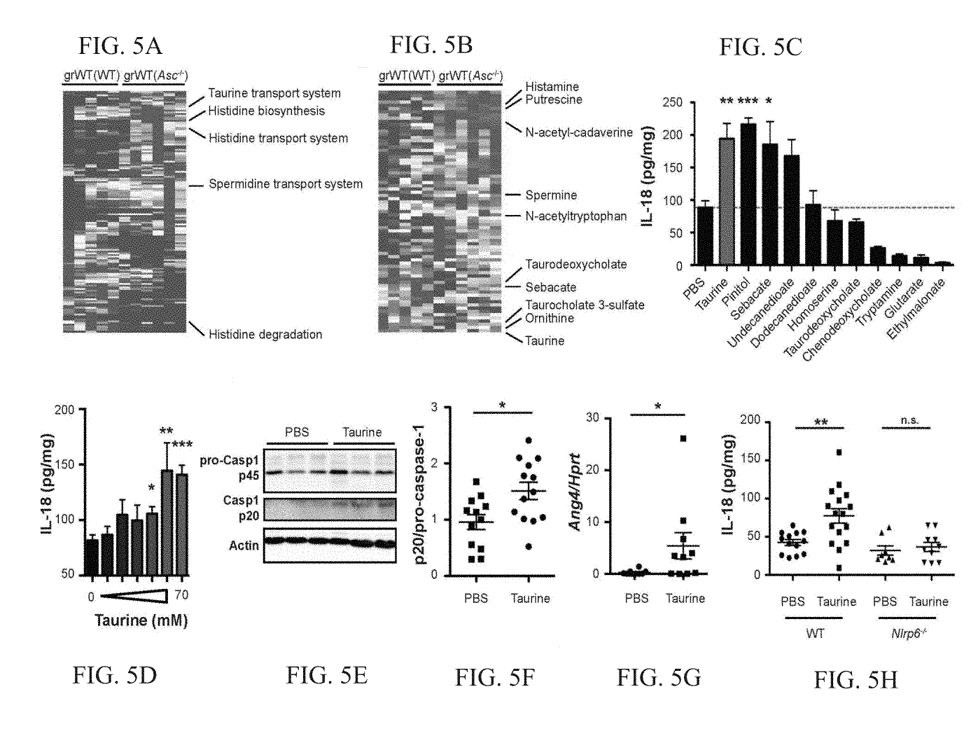

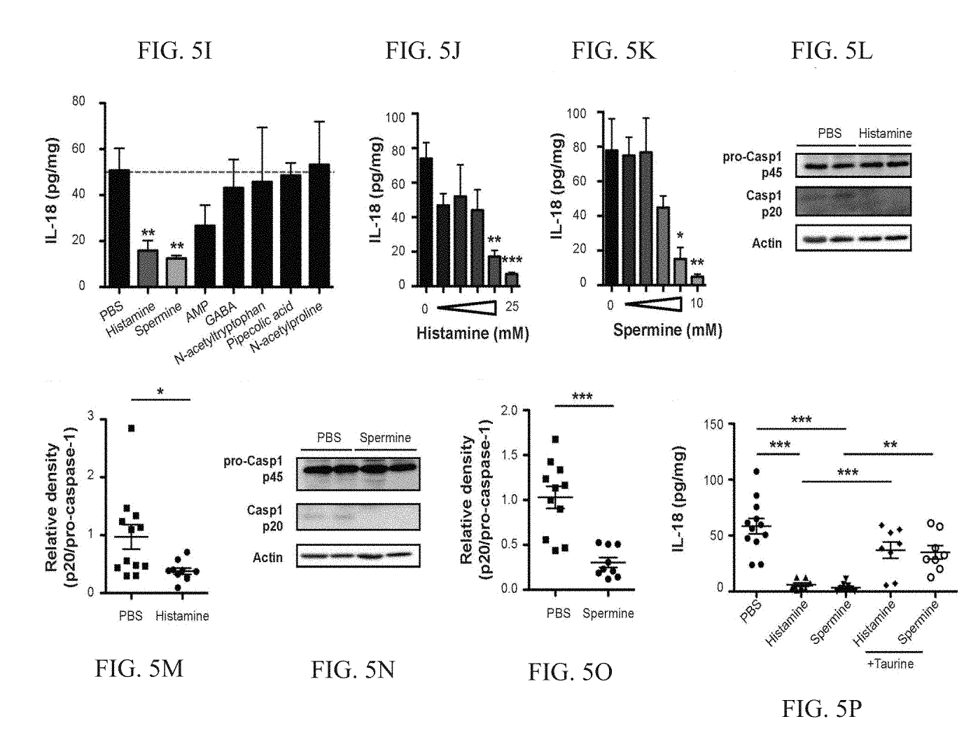

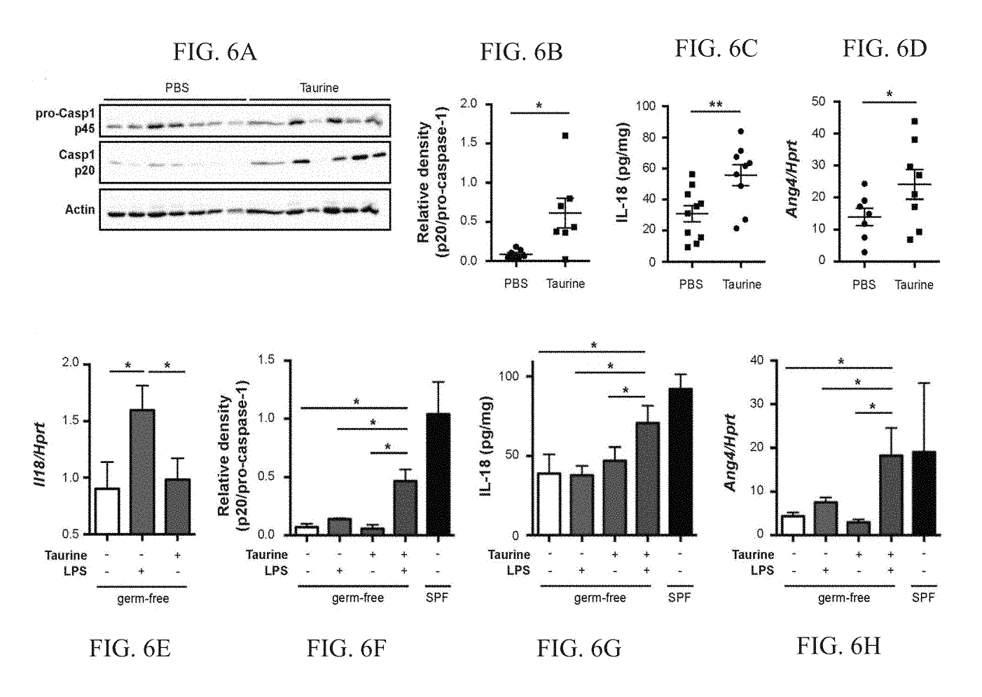

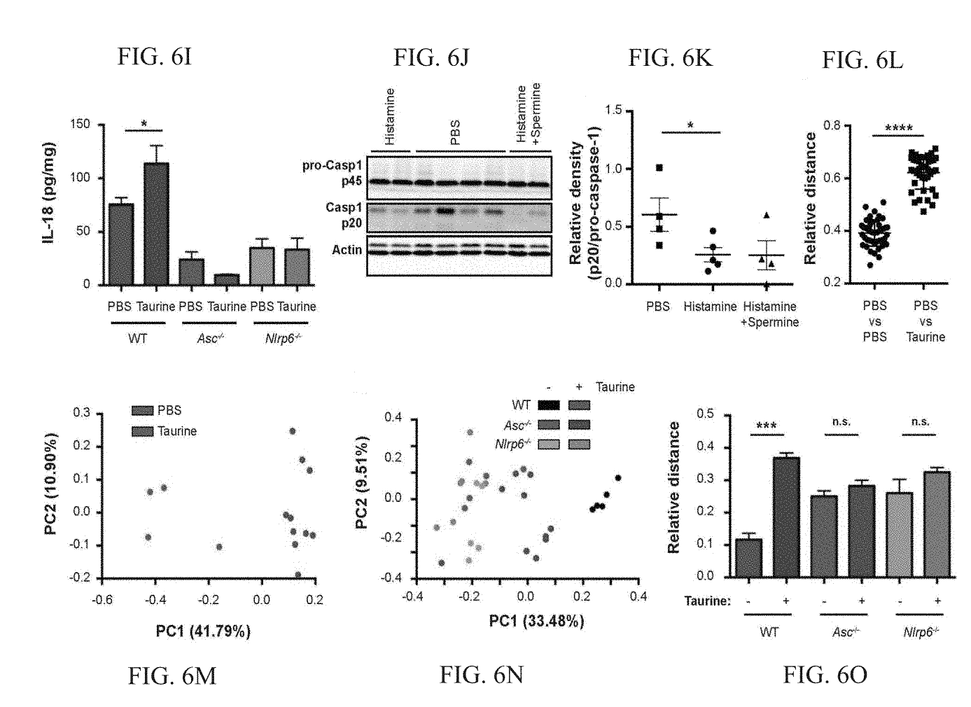

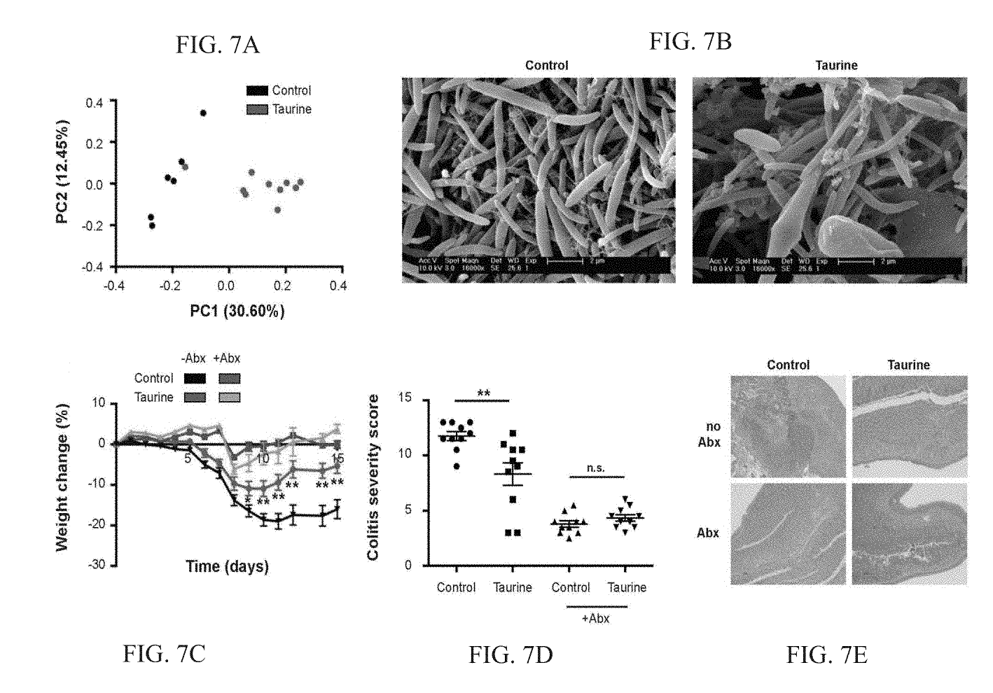

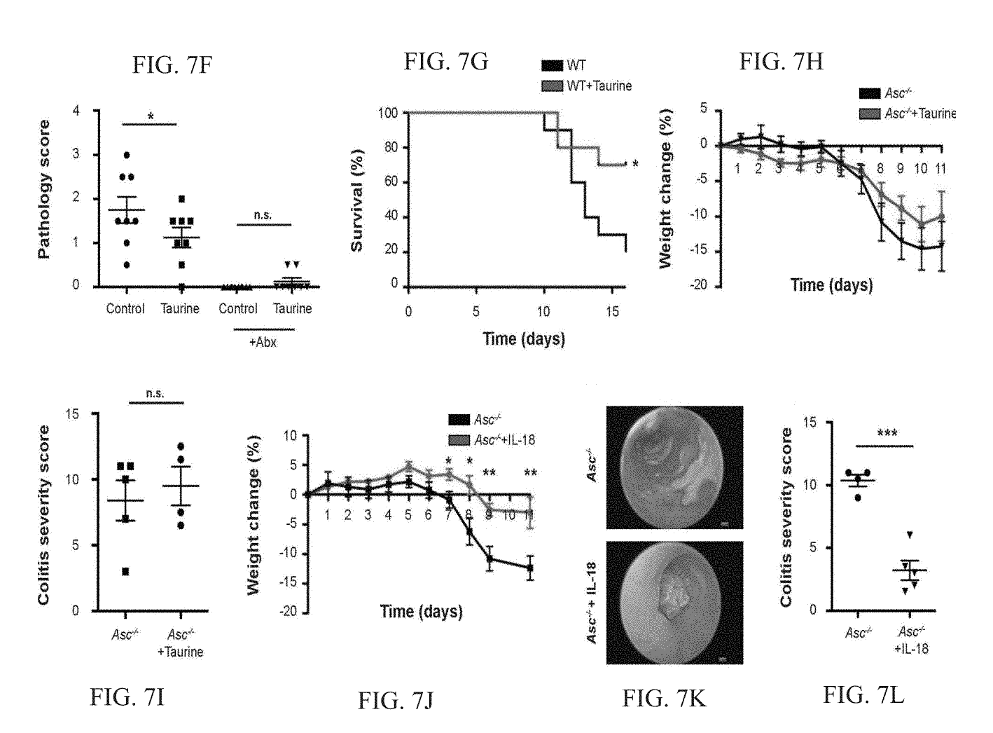

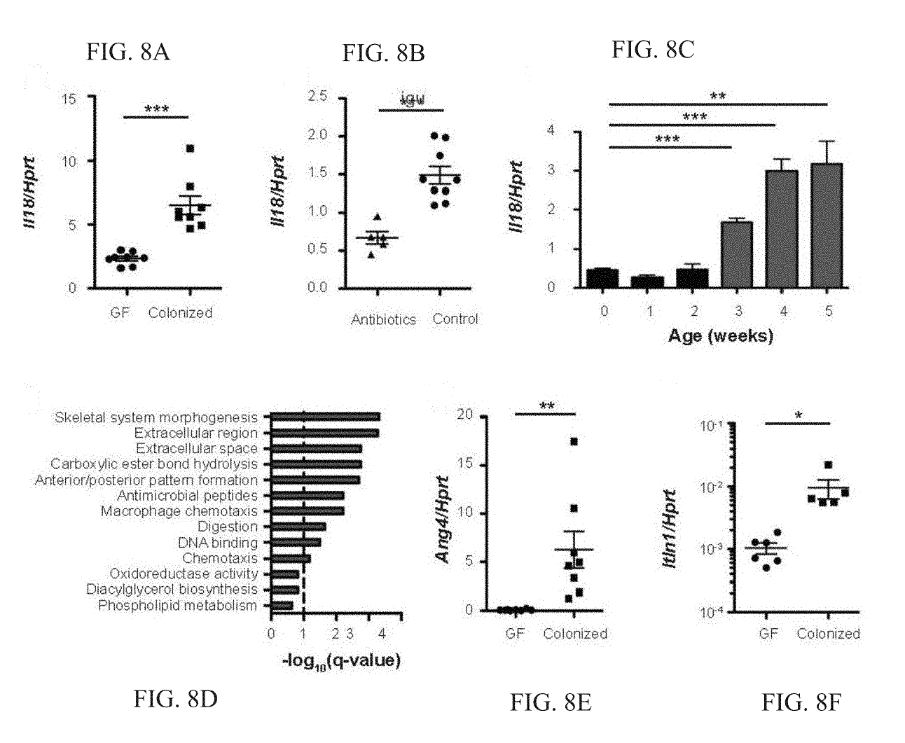

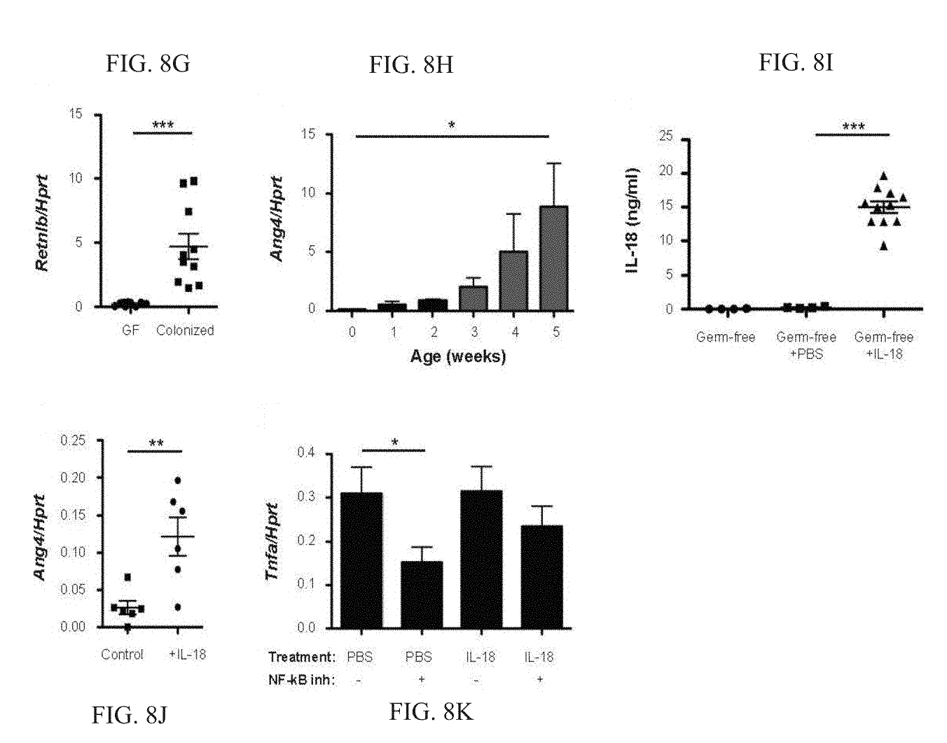

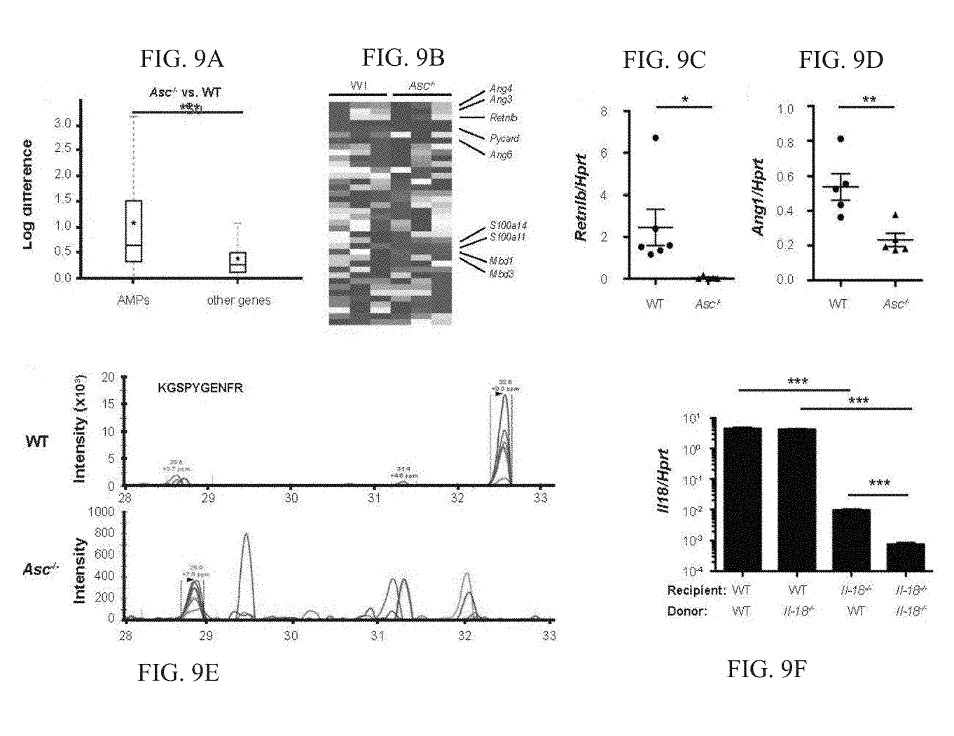

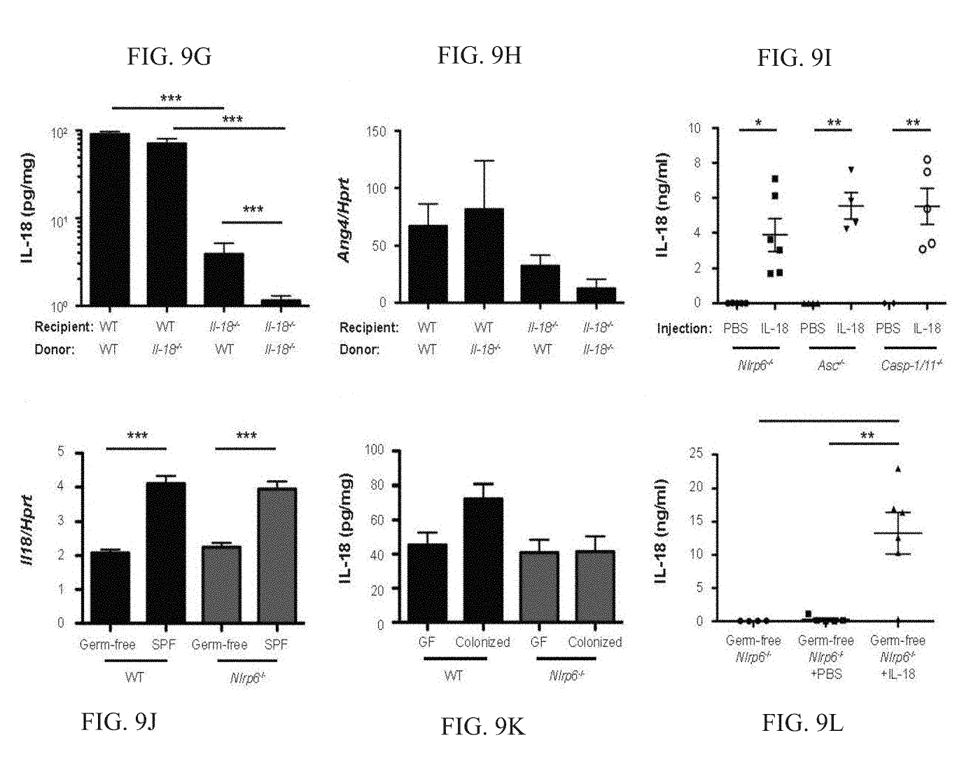

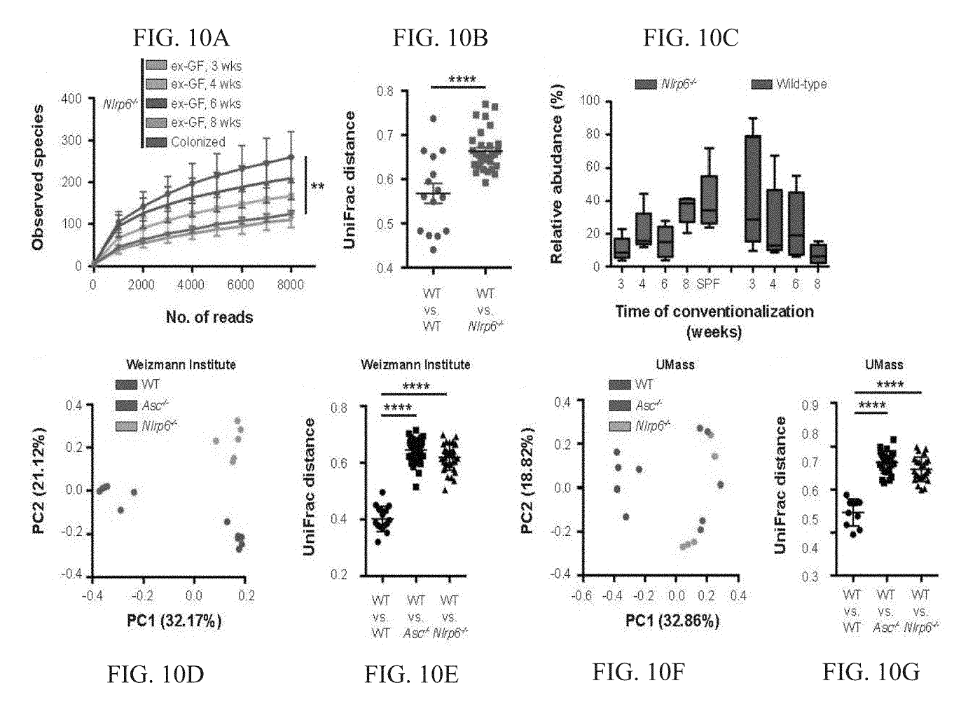

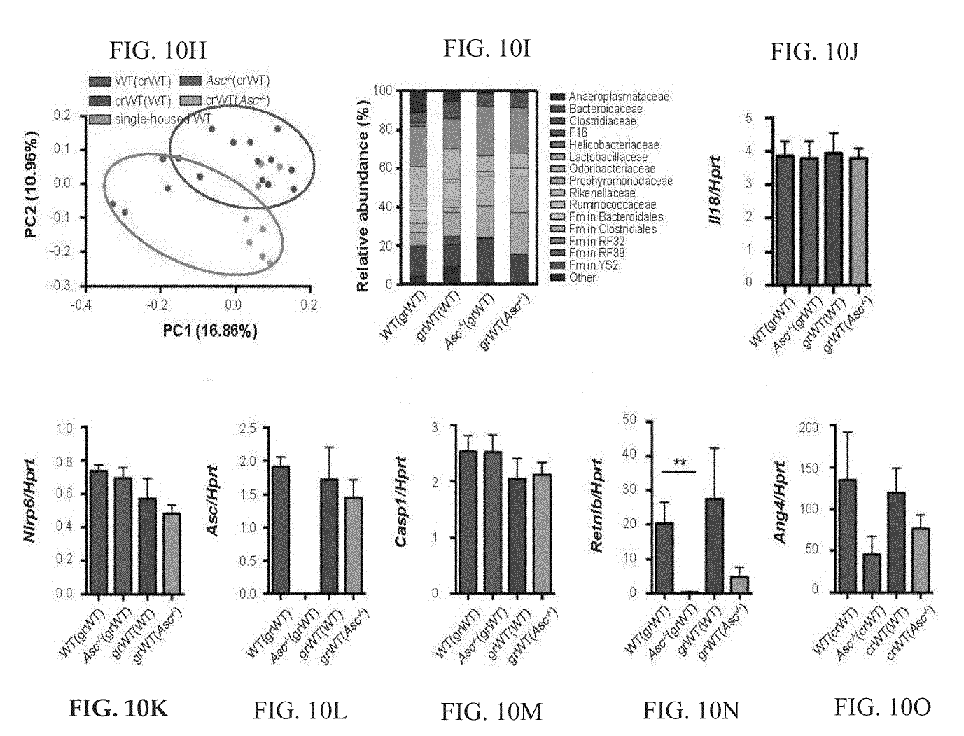

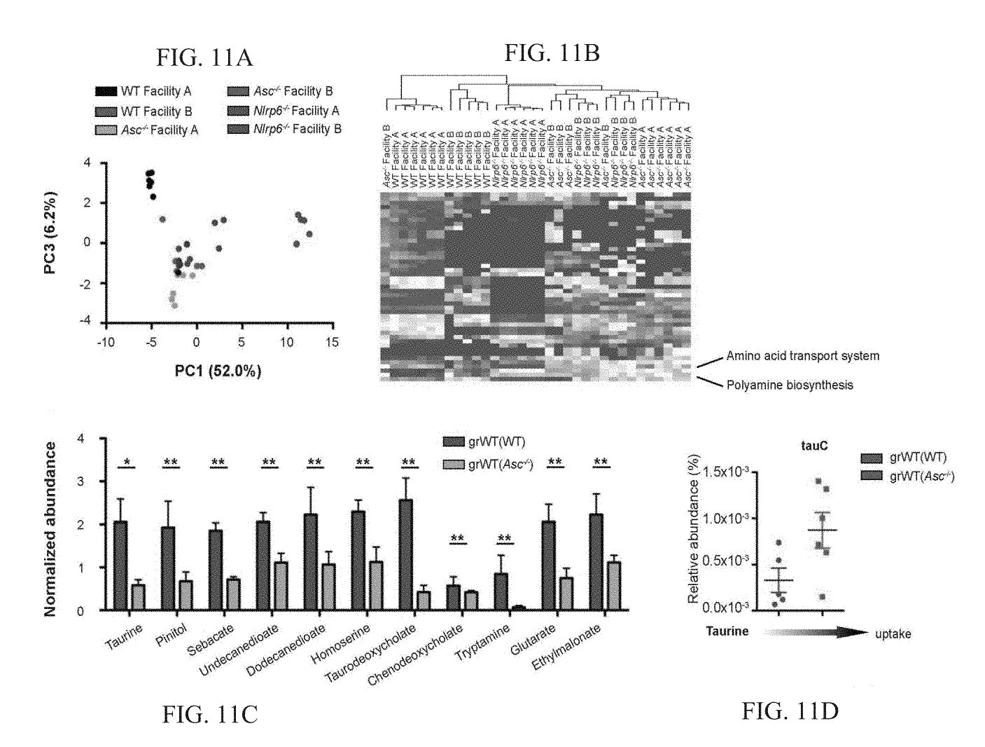

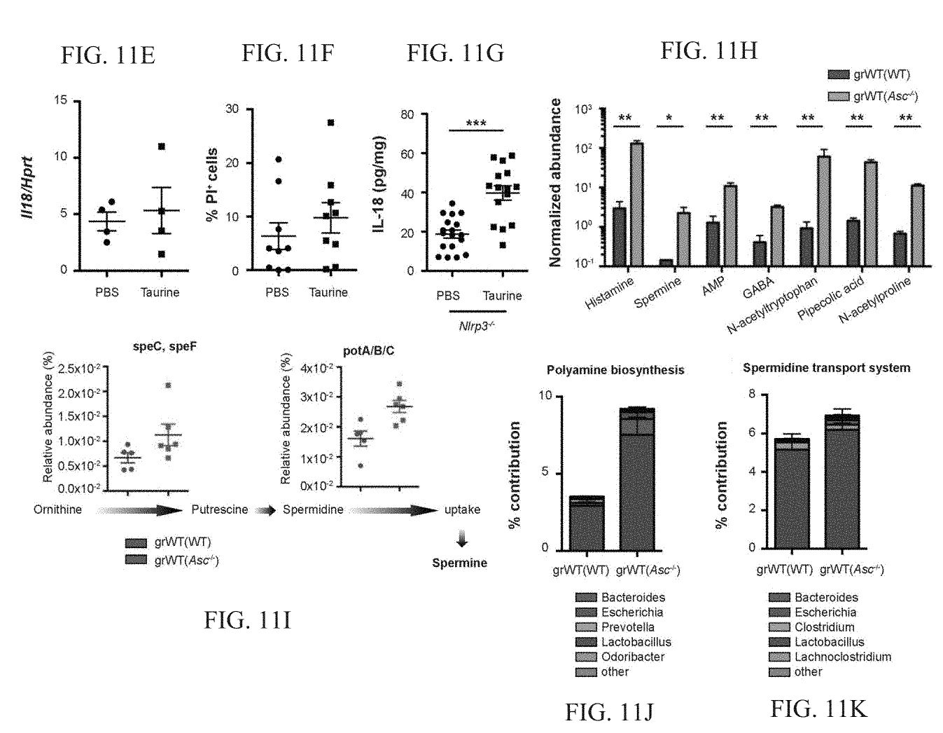

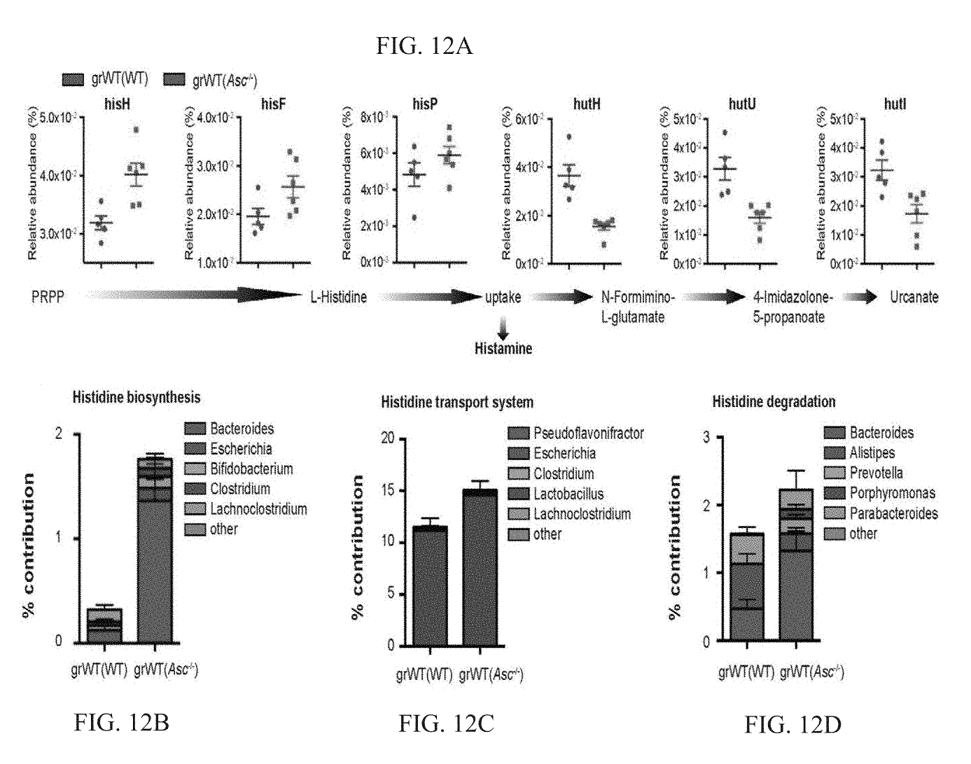

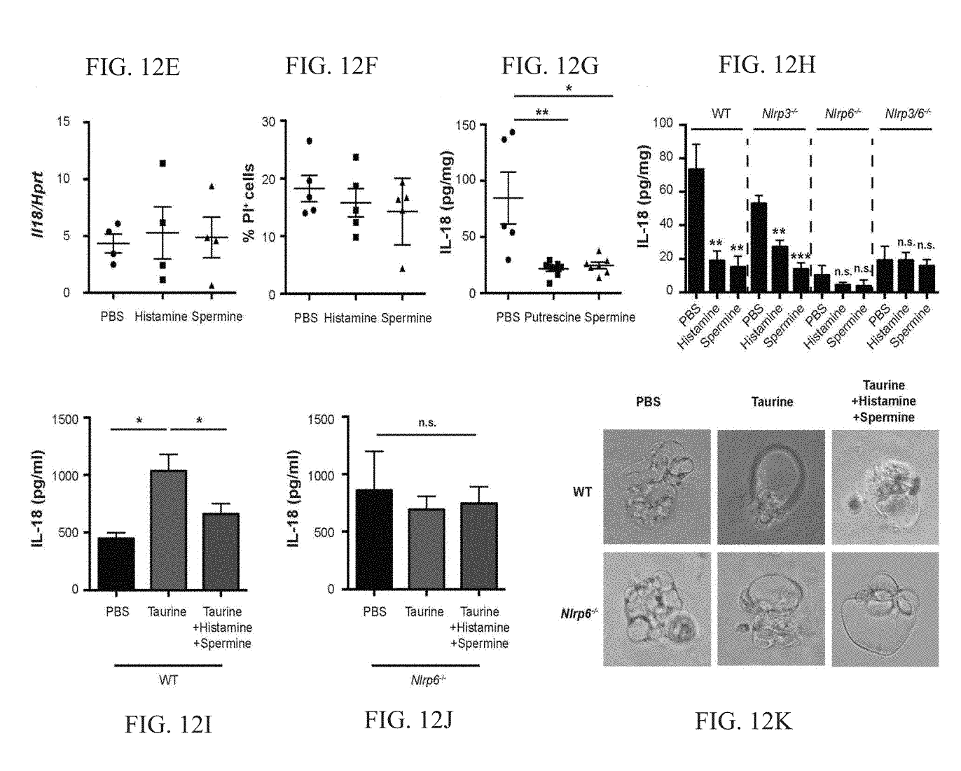

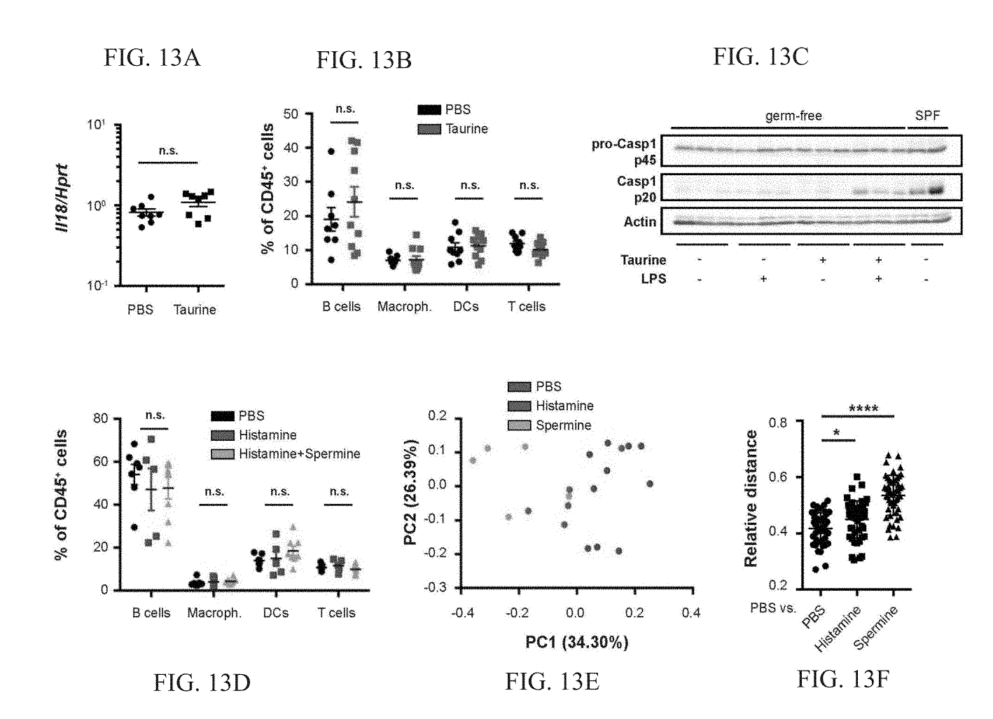

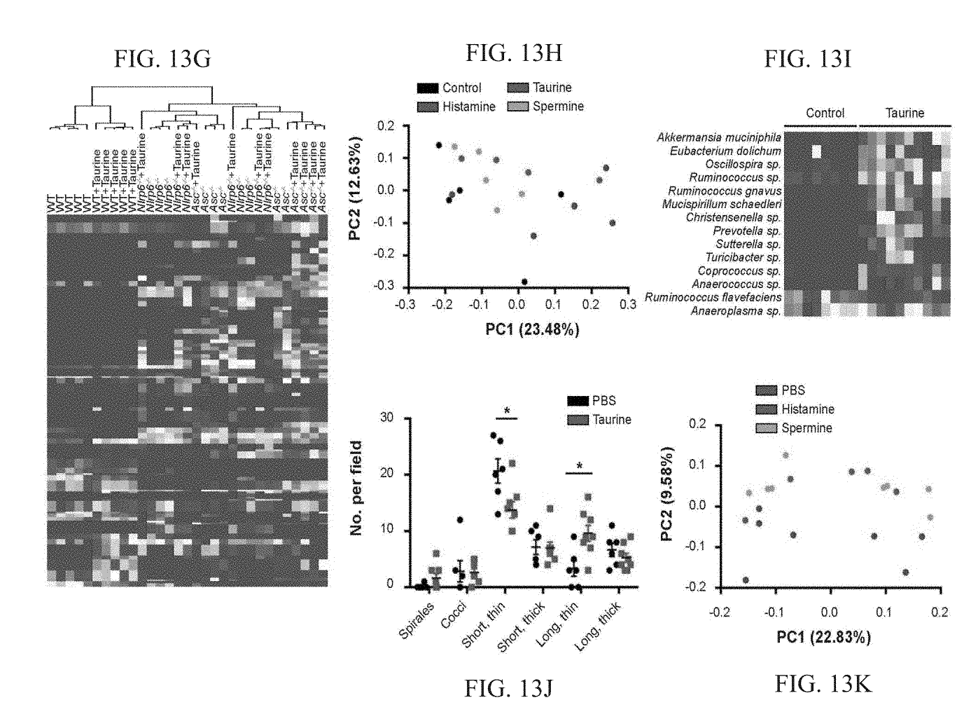

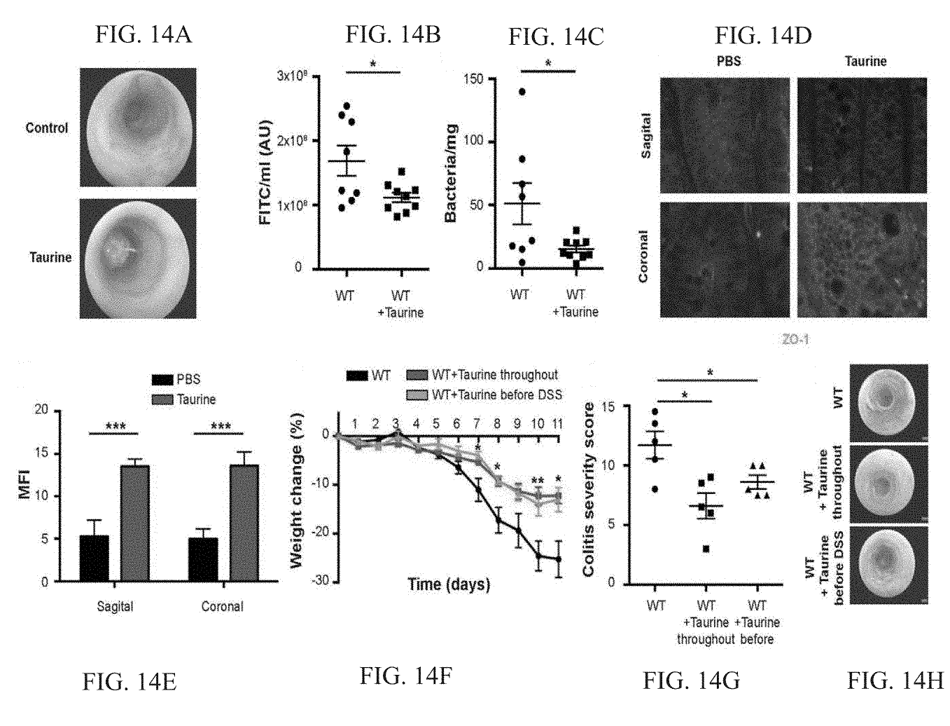

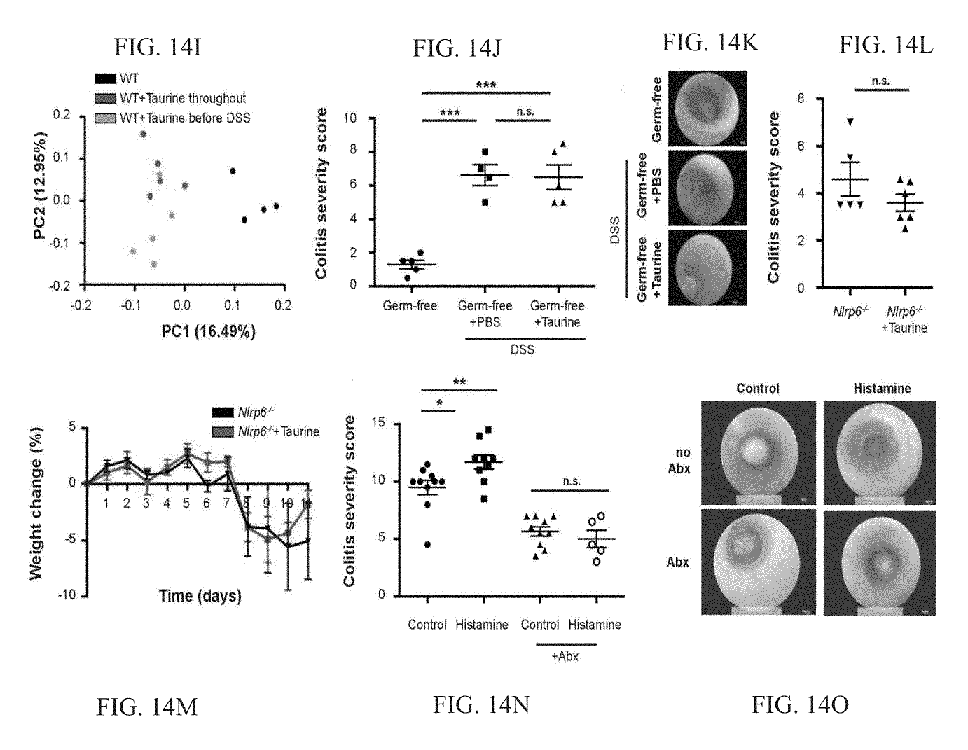

In order to investigate the regulatory principles orchestrating gut microbiota niche formation, the effects of commensal bacterial colonization on inflammasome signaling were studied in the wild-type (WT) setting. The gut microbiota were shown to be critically important for inflammasome activation, as its absence in germ-free (GF) mice resulted in an abolished caspase-1 auto-cleavage (FIGS. 1A and 1B). This severely altered inflammasome activity, coupled with reduced IL-18 mRNA levels (FIG. 8A), resulted in abrogated levels of colonic IL-18 in WT GF mice (FIG. 8C). Similarly, WT mice treated with broad-spectrum antibiotics (vancomycin, ampicillin, neomycin and metronidazole, 1 g/l in drinking water) featured decreased mRNA (FIG. 8B) and protein levels of IL-18 (FIG. 8D). To further corroborate the role of the microbiota in inducing mucosal IL-18, the present inventors examined the levels of IL-18 in the neonatal period, in which all newborn mammals feature progressive intestinal microbial colonization, accompanied by intestinal mucosal barrier formation and immune system maturation (Renz et al., 2012). Indeed, intestinal IL-18 levels progressively increased over the first five weeks of post-natal development, paralleling microbial colonization (FIGS. 1E and 7C, (Kempster et al., 2011)).

The present inventors next determined the consequences of commensal-mediated IL-18 induction on the host gut mucosal niche, by performing global RNA-seq of colonic tissue from WT and Il18^{-/-} mice. They then grouped host transcripts into functional categories and evaluated differential expression of these categories between WT and Il18^{-/-} mice (FIG. 8D). One of the most differentially represented categories included anti-microbial pathways (FIGS. 1F and 1G), suggesting a role for IL-18 in regulating the anti-microbial program of the colonic mucosa. Among the antimicrobial peptides induced by IL-18 were ITLN1, RELMβ, and members of the angiogenin family (FIGS. 1H-1K), for which a microbicidal activity has been previously reported (Artis et al., 2004; Tsuji et al., 2001). Of these, angiogenin-4 (Ang4) expression has been suggested to be influenced by the intestinal microbiota (Hooper et al., 2003). Indeed, GF mice featured nearly undetectable levels of Ang4 (FIG. 8E), as well as ITLN1 and RELMβ (FIGS. 8F and 8G), while microbial colonization during early development progressively induced AMP expression in newborn WT mice (FIG. 8H). Both administration of IL-18 under sterile conditions to GF colon explants and in-vivo administration of IL-18 to WT GF mice (FIG. 8I) restored normal levels of colonic AMPs (FIGS. 1L, 1M, and 8J). IL-18 induction of AMPs was dependent on NF-κB signaling, as demonstrated by NF-κB inhibition, which prevented AMP induction (FIG. 1N and FIG. 8K). Altogether, these results suggest that microbiota-induced colonic IL-18 is both necessary and sufficient for the regulation of intestinal anti-microbial peptide production.

To determine whether the NLRP6 inflammasome drives this IL-18-mediated induction of colonic anti-microbial activity, the present inventors performed global RNA-seq of colonic tissue from WT mice and mice deficient in either the inflammasome adapter ASC or the upstream NLR protein NLRP6. Similar to Il-18^{-/-} mice, Asc^{-/-} and Nlrp6^{-/-} mice featured an abnormal AMP profile, including impaired levels of angiogenins and RELMβ (FIGS. 2A-D), suggesting that control of AMP expression requires an intact NLRP6 inflammasome. This aberrant AMP profile of Asc-deficient mice was also noted by RNA-seq of colonic tissue obtained from mice housed in an independent North American animal facility (FIGS. 9A and 9B). To validate these results, the present inventors further focused on colonic IL-18 and the prototypical AMP Ang4, and compared their levels in WT mice to those in Nlrp6^{-/-}, Asc^{-/-}, Il-18^{-/-}, Casp-1/11^{-/-} and Nlrp3^{-/-} mice. While mice lacking NLRP3 had normal levels of colonic IL-18 and Ang4 levels, Nlrp6^{-/-}, Asc^{-/-}, Il-18^{-/-}, and Casp-1/11^{-/-} mice featured a marked reduction in both IL-18 and Ang4, suggesting that the NLRP6 inflammasome is required for IL-18 production upstream of AMP induction (FIGS. 2E and 2F). Similarly, Asc^{-/-} mice featured low colonic levels of Retn1b and Ang1 (FIGS. 9C-D). Ang4 reduction at the protein level was confirmed by targeted mass-spectrometry for Ang4 peptides (FIGS. 2G and 2H, and FIG. 9E). Bone marrow chimera experiments, using WT or Il-18^{-/-} mice as either donors or recipients of bone marrow transplants, indicated that intestinal IL-18 is primarily produced by the non-hematopoietic compartment (FIGS. 9F and 9G), and that IL-18 originating from this source is necessary for AMP induction (FIG. 9H). In-vivo administration of recombinant IL-18 into mice lacking NLRP6, ASC, or caspase-1/11 restored IL-18 (FIG. 9I) and AMP levels (FIGS. 2I-2M), demonstrating that IL-18 is sufficient for AMP expression downstream of inflammasome signaling.

Since both the microbiota and the NLRP6 inflammasome were required for IL-18 and AMP induction, it was hypothesized that the microbiota may activate NLRP6 upstream of IL-18 secretion. Indeed, while induction of IL-18 transcription in response to microbial colonization functioned normally in the absence of NLRP6 (FIG. 9J), secretion of mature IL-18 protein, and concomitant up-regulation of Ang4 mRNA upon colonization of GF mice were dependent on NLRP6 and severely impaired in conventionalized Nlrp6^{-/-} mice (FIGS. 2N and 9K). Sterile administration of IL-18 into GF Nlrp6^{-/-} mice rescued this defect (FIG. 9L) and induced Ang4 (FIG. 20), indicating that the addition of IL-18 bypasses the need for both the presence of a microbiota and intact inflammasome signaling, and suffices to induce a normal anti-microbial program in response to microbial colonization.

Together, these data uncover a pathway by which the microbiota induces NLRP6 inflammasome signaling to produce IL-18, which in turn activates an AMP program in the colonic mucosal niche.

### The Inflammasome-Antimicrobial Peptide Axis Regulates Intestinal Microbial Community Composition

The present inventors next sought to assess whether the above-identified NLRP6-IL-18-AMP axis is involved in determination of intestinal microbiota composition. Nlrp6^{-/-} mice were recently shown to harbor a dysbiotic microbiome configuration (Elinav et al., 2011). However, whether impaired inflammasome deficiency directly drives dysbiosis (as opposed to cross-generational or facility-related dysbiosis) remained to be investigated. Therefore the temporal microbial composition was analyzed, using 16S rDNA sequencing of GF Nlrp6^{-/-} mice that were allowed to spontaneously conventionalize at the vivarium. Notably, GF Nlrp6^{-/-} mice gradually shifted their microbial community composition towards the dysbiotic configuration of Nlrp6^{-/-} mice that had been housed in their specific pathogen-free (SPF) vivarium for multiple generations (FIGS. 3A and 3B). Two months following colonization, the microbiota composition of ex-GF Nlrp6^{-/-} mice became similar to that of SPF Nlrp6^{-/-} mice. This shift was accompanied by a gradual reduction in alpha-diversity down to the level of SPF Nlrp6^{-/-} mice (FIG. 10A). The dysbiotic community development in newly colonized Nlrp6^{-/-} mice was different from that of conventionalized WT GF mice, resulting in a distinct community composition as early as three weeks post microbiota exposure (FIGS. 3C and 10B). The different bacterial community composition is exemplified by the abundance development of Bacteroidaceae (FIG. 10C). To rule out potential confounding effects of animal housing conditions, the present inventors also profiled the microbiota of Nlrp6^{-/-} and Asc−/− mice housed in a vivarium located at a different continent (North America). Dysbiosis occurred independently of animal facilities and housing conditions (FIGS. 10D-G), underlining the critical role of the NLRP6 inflammasome in maintaining a healthy microbial composition in the intestine.

To determine whether the reduction of intestinal IL-18 and AMP levels is responsible for the inability of Nlrp6^{-/-} mice to control microbial community development, recombinant IL-18 was administered to WT or Nlrp6^{-/-} mice and the changes in bacterial composition were determined using 16S rDNA sequencing. While the microbiota of WT mice was not affected by the injection of IL-18, the microbial community of Nlrp6^{-/-} mice underwent marked compositional changes (FIG. 3D). Similarly significant configuration changes upon IL-18 administration were observed in Asc- and Caspase-1/11-deficient mice (FIGS. 3E and 3F). Importantly, addition of IL-18 to Caspase-1/11^{-/-} mice corrected the aberrant microbiota composition on the levels of both beta- and alpha-diversity (FIGS. 3G and 3H), and restored the abundance of multiple commensal bacteria back to WT levels (FIG. 3I). Moreover, administration of recombinant Ang4 to anaerobic in-vitro microbiome cultures of Asc^{-/-} mice partially restored beta- and alpha-diversity (FIGS. 3J and 3K), corroborating Ang4 as one of the microbiome-modulating effectors downstream of IL-18. Of note, the impact of Ang4 on the overall community composition of the microbiota from Asc^{-/-} mice was greater than the one derived from WT mice (FIG. 3L), supporting the notion that the absence of AMPs such as Ang4 in Asc^{-/-} mice leads to the outgrowth of normally-suppressed members of the microbiota, which are susceptible to targeting by Ang4 in the in-vitro setting.

Altogether, these data indicate that the identified NLRP6-IL-18-AMP pathway is involved in de-novo determination of the normal intestinal community composition and stability, while its absence drives the development of dysbiosis.

### Dominant Takeover of a Dysbiotic Microbiota Upon Cohabitation is Mediated by Suppression of Inflammasome Activity

One of the hallmark features of the above dysbiotic microbiota configuration in inflammasome-deficient mice is its ability to dominantly transfer to genetically intact mice, where it transmits a variety of inflammatory, metabolic, and neoplastic phenotypes to the WT host (Elinav et al., 2011; Henao-Mejia et al., 2012; Hu et al., 2013). Based on the observation that the orchestration of the mucosal anti-microbial niche structure by IL-18 drives the establishment of a normal or dysbiotic microbiome, the present inventors sought to determine whether this pathway also plays a role in the transmissibility of the aberrant microbiota composition into WT hosts. To this end, colonized WT mice were cohabitated for 4 weeks with WT mice (in which case they were designated colonized recipient, crWT(WT)) or with Asc^{-/-} mice (in which case they were designated as crWT(Asc^{-/-}), FIG. 4A). Cohousing equilibrated the microbiota configuration between cohabitated partners, leading to the establishment of dysbiosis in the genetically intact crWT(Asc^{-/-}) mice (FIG. 9H). Surprisingly, colonic IL-18 levels in recipient WT mice cohoused with Asc^{-/-} mice (crWT(Asc^{-/-})) were significantly lower than those of recipient WT mice cohoused with WT mice (crWT(WT)), similarly to the lower IL-18 levels noted in the donor Asc^{-/-} mice as compared to the donor WT mice (FIG. 4B). These results suggested that the dysbiotic microbiota from inflammasome-deficient mice suppresses IL-18 levels in the WT recipients.

To enable a simplified transmissibility system that would facilitate elucidation of the dysbiotic microbiota-mediated inflammasome suppressive effect on the genetically intact host, GF WT mice were cohabited for 4 weeks with either WT mice (designated germ-free recipient, grWT(WT)) or Asc^{-/-} mice (designated grWT(Asc^{-/-})).

Expectedly, this led to a full transfer of their respective microbiota composition and diversity to the cohoused GF partners (FIGS. 4C, 4D, and 10I), so that both the WT and dysbiotic composition stably persisted in the genetically intact, previously GF mice. Strikingly, and similar to the above observation in conventionalized co-housed WT mice, colonic IL-18 levels in recipient ex-GF WT mice cohoused with Asc^{-/-} mice (grWT(Asc^{-/-})) were as low as in their inflammasome-deficient cohousing partners, while recipient ex-GF WT mice cohoused with WT mice (grWT(WT)) featured normally-high IL-18 levels, similar to those of their cohoused WT partners (FIG. 4E). These differences in colonic IL-18 protein levels were neither due to alterations in IL-18 transcript levels, nor to transcript levels of any NLRP6 inflammasome component, which were unaffected by the genotype or microbiota composition (FIGS. 10J-10M). Instead, caspase-1 processing was abrogated in grWT(Asc^{-/-}) mice as compared to grWT(WT) mice (FIGS. 4F and 4G), indicating that the dysbiotic microbiota originating from Asc^{-/-} mice suppressed intestinal inflammasome activation in the new, genetically intact host. This reduction in inflammasome signaling and IL-18 production was accompanied by an alteration in the global anti-microbial transcriptional program of the colonic mucosa (FIG. 4H), including a strong reduction in Ang4 and RELMβ production (FIGS. 41 and 10N), changes that were also observed in experiments in which the cohoused WT partners were previously colonized (FIG. 100). The microbiota-induced reduction in IL-18 and AMPs was functionally involved in the persistence of the dysbiotic state in the invaded WT host, since replenishment of IL-18 into the cohoused partners prevented dominant transmission of the dysbiotic microbiota configuration from inflammasome-deficient mice into WT recipients (FIGS. 4J and 4K), resulting in an increased UniFrac distance between IL-18-injected mice and their cohousing partners (FIG. 4L). Together, these findings demonstrate that the dysbiotic microbiota stemming from inflammasome-deficient mice modulates inflammasome signaling upon transfer to a new WT host. As a consequence, the anti-microbial milieu of the new colonization niche changes to resemble the dysbiotic niche of origin and allows for community persistence of the invading microbiome.

### Microbiota Metabolites Modulate NLRP6 Inflammasome Signaling in the Healthy and Dysbiotic Settings

To determine the mechanism by which the microbiota modulates inflammasome signaling in the WT and dysbiotic states, the present inventors performed shotgun metagenomic sequencing of grWT(WT) and grWT(Asc^{-/-}) mice and found their respective microbiota to feature a large number of differentially abundant functional KEGG modules (FIG. 5A). Many of the altered pathways involved the generation of small metabolites downstream of energy and nutrient metabolism, including amino acid and polyamine metabolism (FIG. 5A). These changes were consistent across vivaria (FIGS. 11A and 11B).

Since metabolites are considered pivotal mediators of host-microbiota communication (Shapiro et al., 2014), we hypothesized that microbiota-modulated metabolites may take part in regulation of NLRP6 inflammasome signaling and downstream anti-microbial pathways. To this end, we performed a metabolomic screen of cecal content of grWT(WT) mice as compared to grWT(Asc^{-/-}) mice. More than 70 metabolites were found to feature significantly differential levels between these genetically identical recipients (FIG. 5B), among them amino acids and polyamines. Metabolites enriched in the grWT(WT) as compared to grWT(Asc^{-/-}) mice, potentially being microbiota-associated inflammasome activators, included the bile acid conjugate taurine, carbohydrates, and long-chain fatty acids (FIG. 11C). These were tested for inflammasome signaling modulation, by culturing with sterile WT colonic explants and measuring their effect on IL-18 induction (FIG. 5C), with taurine featuring the strongest dose-dependent inductive activity (FIG. 5D). Taurine depletion from the intestinal lumen of grWT(Asc^{-/-}) mice correlated with a higher metagenomic abundance of the taurine transport system that is required for taurine uptake into bacterial cells and subsequent conjugation to secondary bile acids (FIG. 11D). In colonic explants, taurine induced IL-18 secretion by triggering intestinal inflammasome activation, and enhanced caspase-1 cleavage (FIGS. 5E and 5F), while neither influencing IL-18 transcript levels (FIG. 11E) nor cell death (FIG. 11F). Consequently, taurine treatment also induced upregulation of AMPs (FIG. 5G). This induction of IL-18 was dependent on NLRP6, but not on NLRP3 (FIGS. 5H and 11G). Together, these results suggest that taurine is a microbiota-dependent positive inflammasome modulator responsible for enhanced NLRP6 inflammasome-induced IL-18 secretion upon intestinal microbial colonization.

The present inventors next sought to identify metabolites that are involved in the microbiota-induced suppression of inflammasome signaling upon dysbiosis transfer into a WT host. To this end, they focused on metabolites enriched in grWT(Asc^{-/-}) as compared to grWT(WT) mice (FIG. 11H), and screened the most differentially abundant metabolites as potential inflammasome suppressors using the colonic explant system. The two strongest suppressors of IL-18 secretion were histamine and spermine (FIG. 51), both found to be over-represented in colons of grWT(Asc^{-/-}) (FIG. 11H). The accumulation of spermine in the lumen of grWT(Asc^{-/-}) mice was in line with the metagenomic enrichment of the polyamine biosynthesis and transport pathways that are required for the conversion of ornithine into spermine (FIG. 11I). These differences were mainly accounted for by members of the Lactobacillus genus (FIGS. 11J and 11K). Higher histamine levels in grWT(Asc^{-/-}) mice were in line with both increased histidine biosynthesis and transport pathways, as well as decreased histidine degradation (FIG. 12A). A number of bacterial genera contributed to these histamine-related pathways (FIGS. 12B-12D). Both histamine and spermine featured concentration-dependent IL-18 suppressive functions in the colonic explant-based validation screen (FIGS. 5J and 5K), while no change in IL-18 mRNA or cell death was observed (FIGS. 12E and 12F). The metabolite putrescine, a metabolite structurally similar to spermine, equally suppressed IL-18 in colon explants system (FIG. 12G).

Intestinal IL-18 suppression mediated by spermine and histamine resulted from a reduction in NLRP6 inflammasome assembly, as indicated by reduced caspase-1 processing (FIGS. 5L-50) and decreased IL-18 protein levels seen in Nlrp3^{-/-}, but not Nlrp6^{-/-} explants (FIG. 12H). Of note, metabolite-induced inflammasome suppression by either spermine or histamine could be rescued by concomitant administration of taurine (FIG. 5P), suggesting that relative in-vitro contribution of metabolites determine the overall activation of the NLRP6 inflammasome and downstream cytokine production. To validate the ex-vivo colonic explant results, we employed colonic spheroids, an organ-like system amenable to long-term culture (Miyoshi and Stappenbeck, 2013). As in explants, taurine administration to WT but not to Nlrp6^{-/-} organoids induced IL-18 secretion (FIGS. 12I and 12J), while not affecting organoid growth or morphology (FIG. 12K). Additional supplementation with histamine and spermine diminished the taurine-mediated increase in IL-18 production (FIGS. 12I and 12J), while likewise not affecting spheroid growth or morphology (FIG. 12K).

To provide in-vivo validation to the above metabolite activities and to further characterize their effects on the host-microbiome interface, we administered taurine to mice in the drinking water. This led to a marked activation of colonic caspase-1 (FIGS. 6A and 6B), IL-18 secretion (FIG. 6C), and epithelial Ang4 production (FIG. 6D), while not affecting IL-18 mRNA levels (FIG. 13A). These metabolite-induced epithelial changes were not accompanied by alterations in major colonic lamina propria hematopoietic cell populations (FIG. 13B). These results suggested that taurine may constitute a microbial ‘signal II’ for the activation of the colonic NLRP6 inflammasome. To test this notion directly, either LPS (a known ‘signal I’), taurine, or both were administered to GF WT mice. Indeed, LPS, but not taurine, potently induced colonic mRNA of Il18 (FIG. 6E). However, caspase-1 activation, IL-18 secretion, and subsequent Ang4 production were only induced when both LPS and taurine were co-administered (FIGS. 6F-6H and 13C). Taurine's ‘signal II’ function required an intact NLRP6 inflammasome, since no IL-18 induction was observed in taurine-administered Asc^{-/-} or Nlrp6^{-/-} mice (FIG. 61). In contrast to taurine, in-vivo administration of histamine and spermine reduced the levels of activated caspase-1 (FIGS. 6J and 6K), while not altering the composition of hematopoietic cells in the lamina propria (FIG. 13D). These data verify the in-vivo effects of the identified metabolites and suggest that the microbial ‘signal II’ for NLRP6 inflammasome activation has distinct molecular identities in the form of microbiota-related metabolites.

Administration of mice with taurine, histamine, or spermine in drinking water induced compositional changes in the intestinal microbiota (FIGS. 6L, 6M, 13E and 13F), which did not occur upon taurine administration to Asc^{-/-} or Nlrp6^{-/-} mice (FIGS. 6N, 60, and 13G). Anaerobic microbiota cultures supplemented with taurine, histamine, or spermine did not feature significant compositional alterations (FIG. 13H), further indicating that the metabolites do not act directly on commensal bacteria, but required signaling through the host to alter microbial ecology. Metabolite treatment also induced pronounced compositional changes in the epithelial-adherent microbiota, as determined by 16S sequencing and electron microscopy (FIGS. 7A, 7B, and 13I-13K). Together, these results identify distinct microbiome-modulated metabolites as in-vitro and in-vivo regulators of the NLRP6 inflammasome and downstream control of microbiota composition.

### Restoration of the Inflammasome-Antimicrobial Peptide Axis Ameliorates Colitis

Finally, the present inventors sought to determine whether the identification of the metabolite-IL-18-AMP axis has functional significance in disease settings, with a focus on inflammatory bowel disease (IBD). This auto-inflammatory disorder is driven by an impaired host-microbiota niche (Huttenhower et al., 2014), and mediated by a dysbiotic microbiota configuration in inflammasome deficient mice (Elinav et al., 2011).

To determine the potential of the identified metabolites to ameliorate colonic auto-inflammation, taurine was administered in the drinking water to naïve WT mice for two weeks and dextran sodium sulphate (DSS) colitis was induced. Taurine-treated mice featured improved weight loss (FIG. 7C), reduced colitis severity (FIGS. 7D-7F and 14A (Shimizu et al., 2009; Zhao et al., 2008)), enhanced survival (FIG. 7G), and improved mucosal barrier integrity as indicated by a reduced systemic FITC-dextran influx, decreased hepatic bacterial load, and sustained epithelial tight junction integrity (FIGS. 14B-14E). Importantly, taurine's beneficial effects were also observed when taurine administration was stopped before induction of DSS colitis (FIGS. 14F-141), suggesting that the microbial changes, rather than any direct anti-inflammatory effects, were responsible for the amelioration of auto-inflammation. Taurine failed to have beneficial effects when administered to WT mice treated with broad-spectrum antibiotics (FIGS. 7C-7F), GF WT mice (FIGS. 14J and 14K), or mice lacking either ASC or NLRP6 (FIGS. 7H, 7I, 14L, and 14M), suggesting that its activity requires an intact NLRP6 inflammasome and presence of the microbiota. In contrast to taurine, histamine treatment exacerbated DSS colitis in WT mice, an effect that was prevented when mice were concomitantly treated with antibiotics (FIGS. 14N and 140), further underlining the microbiome-dependency of the metabolite-mediated modulation of inflammation.

As inflammasome-deficient mice were resistant to taurine-mediated improvement of colitis, the present inventors tested whether an intervention targeting host signaling downstream of the NLRP6 inflammasome would improve disease severity in these mice. To this aim, they systemically (i.p) administered twice-daily IL-18 or vehicle control to Asc^{-/-} mice for two weeks, followed (after cessation of IL-18 treatment) by induction of DSS colitis. Indeed, administration of IL-18 prior to induction of colonic inflammation diminished colitis severity in Asc^{-/-} mice, as assessed by reduced weight loss (FIG. 7J) and an improved colonoscopy score (FIGS. 7K and 7L). Together, these results suggest that metabolite administration can modulate inflammasome signaling and downstream microbial composition, host physiology, and disease susceptibility.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a disease in a subject in need thereof comprising:
(a) analyzing metabolites of the microbiome of the subject;
(b) identifying the metabolites that are differentially produced in the microbiome of the subject as compared with the metabolites produced in a microbiome of a healthy subject; and
(c) administering to the subject a therapeutically effective amount of at least one metabolite which is down-regulated in the microbiome of the subject as compared with a microbiome of a healthy subject or administering to the subject a therapeutically effective amount of an agent which down-regulates a metabolite which is up-regulated in the microbiome of the subject as compared with a healthy subject, thereby treating the disease.

2. A method of monitoring a treatment of a disease in a test subject:
(a) treating the test subject with at least one metabolite that is produced in a microbiome of the subject;
(b) analyzing a signature of the microbiome of the test subject; and subsequently
(c) comparing said microbiome signature of the test subject with the microbiome signature of a healthy subject, wherein an increase in the similarity of the microbiome signature of the test subject with the microbiome signature of the healthy subject following the treating as compared to the similarity of the microbiome signature of the test subject with the microbiome signature of the healthy subject prior to the treating is indicative of an effective treatment.

3. The method of claim 2, wherein said signature of the microbiome comprises a metabolite signature of the microbiome.

4. The method of claim 1, further comprising comparing said metabolites that are produced in the microbiome of the subject with the metabolites that are produced in the microbiome of a diseased subject.

5. (canceled)

6. The method of claim 1, wherein said microbiome is a gut microbiome.

7. The method of claim 6, wherein said analyzing is effected in a fecal sample of the subject.

8. The method of claim 1, wherein said analyzing is effected in a blood sample of the subject.

9. A method of treating a disease in a subject in need thereof comprising administering a therapeutically effective amount of at least two metabolites to the subject, wherein the amount of metabolites provided is such that the metabolite signature of the microbiome of the subject is made more similar to the metabolite signature of the microbiome of a healthy subject, thereby treating the disease.

10. The method of claim 9, wherein the metabolite signature comprises a dynamic metabolite signature.

11. A method of treating an inflammatory bowel disease in a subject in need thereof comprising administering to the subject:
(i) a therapeutically effective amount of an agent which down-regulates an amount and/or activity of a metabolite selected from the group consisting of spermine and putrescine; or
(ii) a therapeutically effective amount of taurine and at least one agent which down-regulates an amount and/or activity of a metabolite selected from the group consisting of histamine, spermine and putrescine, thereby treating the inflammatory bowel disease in the subject.

12. (canceled)

13. A method of diagnosing an inflammatory bowel disease in a subject comprising analyzing the amount of a metabolite selected from the group consisting of taurine, histamine, putrescine and spermine produced in the microbiome of the subject, when a decrease in taurine below a predetermined level and/or an increase in histamine, putrescine or spermine above a predetermined level is indicative of the inflammatory bowel disease.

14. The method of claim 1, wherein said disease is an inflammatory disease.

15. The method of claim 1, wherein said disease is a metabolic disease.

16. The method of claim 14, wherein said inflammatory disease is inflammatory bowel disease.

17. The method of claim 16, wherein said inflammatory bowel disease is colitis.

18. The method of claim 16, wherein said inflammatory bowel disease is Crohn's disease.

19. The method of claim 15, wherein said metabolic disease is Diabetes or pre-Diabetes.

20. An article of manufacture comprising taurine and an agent that down-regulates an amount and/or activity of a metabolite selected from the group consisting of histamine, spermine and putrescine.

21. The article of manufacture of claim 20, wherein said taurine and said agent are coformulated in a single composition.

22. The article of manufacture of claim 20, wherein said taurine and said agent are formulated in individual compositions.

23-26. (canceled)
